(19) United States
(12) Patent Application Publication
(10) Pub. No.: US 2025/0195142 A1
(43) Pub. Date: Jun. 19, 2025

(54) TOOL NAVIGATION USING A CUSTOMIZED MEDICAL SIMULATION

(71) Applicant: Intuitive Surgical Operations, Inc., Sunnyvale, CA (US)

(72) Inventors: Gaurav Lamba, Fremont, CA (US); Woojin Ahn, Johns Creek, GA (US); Joey Chau, Cupertino, CA (US)

(73) Assignee: Intuitive Surgical Operations, Inc., Sunnyvale, CA (US)

(21) Appl. No.: 18/981,234

(22) Filed: Dec. 13, 2024

Related U.S. Application Data
(60) Provisional application No. 63/610,761, filed on Dec. 15, 2023.

(51) Int. Cl. A61B 34/00; A61B 34/10; A61B 34/20

(52) U.S. Cl. CPC A61B 34/00; A61B 2034/105; A61B 2034/107; A61B 2034/2065

(57) ABSTRACT

Tool navigation using customized medical simulation is provided. A system accesses a 3-dimensional model of an organ generated via a scan of a subject on which a procedure is to be performed via a robotic medical system. The system registers the 3-dimensional model of the organ with predetermined coupling points in a digital environment established for the procedure on the subject. The system executes, via the digital environment, a simulation of the procedure to identify a candidate path for a tool through the 3-dimensional model of the organ registered with the predetermined coupling points in the digital environment. The system provides, for display via a graphical user interface, an indication of the candidate path for the tool to perform the procedure via the robotic medical system.

### CROSS-REFERENCES TO RELATED APPLICATIONS

This application claims the benefit of priority under 35 U.S.C. § 119 to U.S. Provisional Patent Application No. 63/610,761, filed Dec. 15, 2023, which is hereby incorporated by reference herein in its entirety.

### BACKGROUND

Medical procedures can be performed in a medical environment on a subject. To facilitate performance of the medical procedure, a surgeon can practice the medical procedure in a digital simulation of an aspect of the medical environment. However, due to the complexity associated with certain medical procedures and the variations of various anatomies from subject to subject, and the computing resource intensive nature of generating an accurate and reliable digital simulation for a medical procedure on a particular subject, it can be technically challenging to generate a digital simulation for a particular procedure on a particular subject without excessive computing resource utilization or the introduction of latencies or delays.

### SUMMARY

Technical solutions disclosed herein are generally related to providing tool navigation using customized medical simulations. For example, this disclosure can incorporate patient-specific 3-dimensional (“3D”) models into a procedural simulation module to create a patient specific simulation experience. The patient-specific 3D model can be generated based on a medical scan generated using a medical imaging technique (e.g., a computed tomography (“CT”) or magnetic resonance (“MR”) imaging). Using this patient-specific 3D model of an organ, this technology can facilitate a more realistic simulation of a procedure in a digital simulation environment. A surgeon can generate one or more candidate tool paths during the simulation, and evaluate the performance of the various candidate tool paths before selecting a path for use during the surgery. The system can provide the simulated tool path for use in various ways during the procedure.

Aspects of the technical solutions are directed to a system. The system can include one or more processors, coupled with memory. The one or more processors can access a 3-dimensional (“3D”) model of an organ of a subject on which a procedure is to be performed via a robotic medical system. The 3D model can be generated via a scan of the subject. The one or more processors can register the 3-dimensional model of the anatomical structure with predetermined coupling points in a digital environment established for the procedure on the subject. The one or more processors can execute, via the digital environment, a simulation of the procedure. The one or more processors can execute the simulation to identify a candidate path for a tool through the 3-dimensional model of the anatomical structure registered with the predetermined coupling points in the digital environment. The one or more processors can provide, for display via a graphical user interface, an indication of the candidate path for the tool to perform the procedure via the robotic medical system.

In some aspects, the one or more processors can access a 3-dimensional model of the subject generated with a first resolution. The 3-dimensional model of the anatomical structure can be generated with a second resolution that is greater than the first resolution. The one or more processors can register, via the predetermined coupling points, the 3-dimensional model of the anatomical structure with the 3-dimensional model of the subject in the digital environment.

The one or more processors can receive an indication of one or more portions of the subject on which the procedure is to be performed. The one or more processors can identify a resolution of one or more 3-dimensional models for the one or more portions of the subject based on the indication that the procedure is to be performed on the one or more portions. The one or more processors can select the 3-dimensional models having the resolution for registration in the digital environment.

In some aspects, the predetermined coupling points can include a first vein and a second vein. The one or more processors can align a first point on the 3-dimensional model of the anatomical structure with the first vein in the digital environment. The one or more processors can align a second point on the 3-dimensional model of the anatomical structure with the second vein in the digital environment.

The one or more processors can adjust a size of a simulated anatomy in the digital environment to register the 3-dimensional model of the anatomical structure in the digital environment. The one or more processors can adjust an orientation of the 3-dimensional model of the organ to register the 3-dimensional model of the anatomical structure in the digital environment.

The one or more processors can identify, upon execution of the simulation of the procedure, a plurality of candidate paths for the tool through the 3-dimensional model. The plurality of candidate paths can include the candidate path. The one or more processors can determine, based on the simulation, a first value for a performance metric for the candidate path of the plurality of candidate paths. The one or more processors can determine, based on the simulation, a second value for the performance metric for a second candidate path of the plurality of candidate paths. The one or more processors can select the candidate path based on a comparison of the first value and the second value. The one or more processors can provide, responsive to the selection, the indication for the candidate path for display via the graphical user interface.

The one or more processors can identify, upon execution of the simulation of the procedure, a value of a performance metric related to the candidate path for the tool to perform. The one or more processors can identify a threshold for the performance metric based on at least one of a type of procedure or a type of the anatomical structure. The one or more processors can provide, responsive to the value of the performance metric satisfying the threshold, the candidate path for display via the graphical user interface.

The one or more processors can display, via the graphical user interface, one or more values of the performance metric in association with one or more candidate paths. The one or more processors can overlay, via the graphical user interface, the indication of the candidate path on the 3-dimensional model of the anatomical structure.

The one or more processors can receive a video stream captured by a camera of the procedure performed via the robotic medical system on the subject in a medical environment. The one or more processors can display the video stream via the graphical user interface. The one or more processors can overlay the indication of the candidate path for the tool through the anatomical structure on the video stream of the procedure displayed via the graphical user interface.

The one or more processors can receive, subsequent to the display of the candidate path, a command to reject the candidate path displayed via the graphical user interface. The one or more processors can remove, responsive to the command, the display of the candidate path. The one or more processors can display, responsive to the request, a second candidate path identified during execution of the simulation.

The one or more processors can receive, via one or more sensors, a data stream of the procedure performed via the robotic medical system on the subject in a medical environment. The one or more processors can determine a first value of a performance metric associated with the procedure performed via the robotic medical system on the subject in the medical environment. The one or more processors can determine a second value of the performance metric related to the candidate path identified during execution of the simulation. The one or more processors can provide a notification via the graphical user interface based on a comparison of the first value and the second value.

The one or more processors can generate a value of a second performance metric based at least in part on the comparison of the first value and the second value. The one or more processors can display, via the graphical user interface, the value of the second performance metric. In some cases, the one or more processors can display a location of the tool during the procedure performed via the robotic medical system on the subject in the medical environment. The one or more processors can display the location of the tool on the graphical user interface comprising the indication of the candidate path for the tool to perform the procedure via the robotic medical system.

The one or more processors can determine a distance between the location of the tool and the candidate path for the tool. The one or more processors can provide a second notification responsive to the distance being greater than a threshold. The threshold can be selected based at least in part on the type of the procedure or the type of the anatomical structure.

Aspects of the technical solution are directed to a method. The method can be performed by one or more processors coupled with memory. The method can include the one or more processors accessing a 3-dimensional model of an organ generated via a scan of a subject on which a procedure is to be performed via a robotic medical system. The method can include the one or more processors registering the 3-dimensional model of the anatomical structure with predetermined coupling points in a digital environment established for the procedure on the subject. The method can include the one or more processors executing, via the digital environment, a simulation of the procedure to identify a candidate path for a tool through the 3-dimensional model of the anatomical structure registered with the predetermined coupling points in the digital environment. The method can include the one or more processors providing, for display via a graphical user interface, an indication of the candidate path for the tool to perform the procedure via the robotic medical system.

The method can include the one or more processors accessing a 3-dimensional model of the subject generated with a first resolution, wherein the 3-dimensional model of the anatomical structure is generated with a second resolution that is greater than the first resolution. The method can include the one or more processors registering, via the predetermined coupling points, the 3-dimensional model of the anatomical structure with the 3-dimensional model of the subject in the digital environment.

Aspects of the technical solutions are directed to a non-transitory computer-readable medium storing processor-executable instructions that, when executed by one or more processors, cause the one or more processors to access a 3-dimensional model of an organ generated via a scan of a subject on which a procedure is to be performed via a robotic medical system. The instructions can include instructions to register the 3-dimensional model of the anatomical structure with predetermined coupling points in a digital environment established for the procedure on the subject. The instructions can include instructions to execute, via the digital environment, a simulation of the procedure to identify a candidate path for a tool through the 3-dimensional model of the anatomical structure registered with the predetermined coupling points in the digital environment. The instructions can include instructions to provide, for display via a graphical user interface, an indication of the candidate path for the tool to perform the procedure via the robotic medical system.

An aspect can be directed to a method. The method can be performed by one or more processors coupled with memory. The method can include the one or more processors receiving an indication of a phase of a medical procedure being performed on a subject via a robotic medical system. The method can include the one or more processors performing a lookup in a data repository to identify a plurality of candidate paths generated for the phase via a simulation with a 3D model of an organ of the subject. The method can include the one or more processors providing, for display via a graphical user interface, an indication of the plurality of candidate paths and a metric associated with each of the plurality of candidate paths. The method can include the one or more processors receiving a selection of a candidate path of the plurality of candidate paths. The method can include the one or more processors displaying the selected candidate path to facilitate performance of the phase of the medical procedure on the subject via the robotic medical system.

These and other aspects and implementations are discussed in detail below. The foregoing information and the following detailed description include illustrative examples of various aspects and implementations and provide an overview or framework for understanding the nature and character of the claimed aspects and implementations. The drawings provide illustration and a further understanding of the various aspects and implementations and are incorporated in and constitute a part of this specification. The foregoing information and the following detailed description and drawings include illustrative examples and should not be considered as limiting.

### BRIEF DESCRIPTION OF THE DRAWINGS

The accompanying drawings are not intended to be drawn to scale. Like reference numbers and designations in the various drawings indicate like elements. For purposes of clarity, not every component can be labeled in every drawing. In the drawings:

FIG. 1 depicts an example system of tool navigation using a customized medical simulation.

FIG. 2A depicts an example method of tool navigation using a customized medical simulation.

FIG. 2B depicts example graphical user interface visualization for tool navigation using a customized medical simulation.

FIG. 3 depicts an example method of tool navigation using a customized medical simulation.

FIG. 4 depicts an example method of tool navigation using a customized medical simulation.

FIG. 5 depicts an example medical environment.

FIG. 6 is a block diagram depicting an architecture for a computer system that can be employed to implement elements of the systems and methods described and illustrated herein, including aspects of the system depicted in FIG. 1, and the methods or processes depicted in FIGS. 3 and 4.

### DETAILED DESCRIPTION

Following below are more detailed descriptions of various concepts related to, and implementations of, methods, apparatuses, and systems of tool navigation using a customized medical simulation. The various concepts introduced above and discussed in greater detail below can be implemented in any of numerous ways.

Although the present disclosure is discussed in the context of a surgical procedure, in some embodiments, the present disclosure can be applicable to other medical sessions or environments or activities, as well as non-medical activities where removal of irrelevant information is desired.

Technical solutions disclosed herein are generally related to providing tool navigation using customized medical simulations. For example, this disclosure can incorporate patient-specific 3D models into a procedural simulation module to create a patient specific simulation experience. The patient-specific 3D model can be generated based on a medical scan generated using a medical imaging technique, such as CT or MR scans. Using this patient-specific 3D model of an organ, this technology can facilitate a more realistic simulation of a procedure in a digital simulation environment. A surgeon can generate one or more candidate tool paths during the simulation, and evaluate the performance of the various candidate tool paths before selecting a path for use during the surgery. The system can provide the simulated tool path for use in various ways during the procedure.

A procedural simulation that is generated and executed without taking patient specific anatomical characteristics may not accurately or reliably represent the medical procedure to be performed on a patient. For example, the simulated anatomical structures may be predetermined, static, or have default characteristics such as shape or volume. As such, the parameters determined or generated during the simulation for input into a robotic medical system may be erroneous or not sufficiently representative of the parameter to be used when performing the medical procedure. The parameters can include, for example, a tool navigation path through an organ, angle of attack or entry, type of tool, duration, order or type of tasks, or objective performance indicators or metrics. Thus, the erroneous, inaccurate, or non-representative simulation may result in the generation of parameters that introduce inefficiencies, delays, or wasted resource utilization in a medical procedure performed by a robotic medical system.

For example, in a partial nephrectomy, a procedural simulation can show a standard kidney, tumor and other anatomical structures. While there may be some parameterization of the anatomical structure (e.g., to make some adjustments to anatomy size and shape), such adjustments are limited and may result in an unreliable representation of a specific patient's kidney, tumor size and share, or vasculature. Thus, it can be challenging to accurately and reliably determine parameters to input into a robotic medical system to perform the medical procedure, which can result in an ineffective or inefficient medical procedure.

Aspects of this technology can include a data processing system that can create a 3D model based on a medical image of a patient or subject. The data processing system can recreate a 3D model based on a CT or MR scan of a patient. The data processing system can use the 3D model to facilitate pre-operation planning, intra-operative reference, or post-operative case review, for example. The 3D model can be specific to the patient since this technology can create the 3D model from the CT or MR scan of the patient. The 3D model can include critical anatomical structures that facilitate planning or the determination or identification of parameters to be used by the robotic medical system to perform the medical procedure. For partial nephrectomy, these anatomical structures can include a kidney, tumor, ureter, collecting structures, arteries and veins, for example. The technology can use a collection of these anatomical structures to generate a digital simulation that can provide a surgeon an informative view of the actual patient anatomy, which helps in preoperative planning, practicing the procedure, or intraoperative decision making.

The data processing system can incorporate the 3D model into The 3D model can be incorporated into various extended reality (“XR”) environments to provide a simulator. To do so, the data processing system can download the 3D model onto a medical simulation console. The technology can register or overlay the 3D model onto simulation anatomy. In some cases, a surgeon can use hand controls of a surgeon console to move, resize, or rotate the model to achieve an overlay. In some cases, the technology can automatically register the 3D model to achieve the overlay. For example, in the simulation environment, the data processing system can use measurements of the simulated critical structures (e.g., kidney) to resize the 3D model to overlay the 3D model of the organ onto the simulated anatomy. Once the data processing system achieves overlay of a larger structure, such as a kidney (which contains other structures like tumor, vessels, or collecting system), the data processing system may proceed to visualization or allow for manual adjustments to be made.

Upon overlaying or registering the patient specific 3D model of an anatomical structure onto the simulated anatomy of the patient, the data processing system can simultaneously provide, via a graphical user interface, a visualization of the simulated anatomy and the 3D model. The data processing system can utilize various settings for the visualization of the 3D model. For example, the data processing system can adjust the transparency of the 3D model, or various substructures of the 3D model. In some cases, the data processing system can receive an instruction from a user to toggle the visibility to on or off for the 3D model or substructures thereof.

The data processing system can provide a simulation. The simulation can include one or more component or aspects of a medical environment. The simulation can include a simulated anatomy of a subject that is overlayed with the 3D model of an anatomical structure, such as an organ. The data processing system can execute the simulation of the medical procedure. The simulation can be interactive. For example, during the simulation, a surgeon can simulate performing a dissection on the simulated anatomy based on the 3D model. In some cases, the 3D model can replace the surgical anatomy, which may have a technical advantage as it can be challenging to generate a simulated 3D anatomy that matches the 3D model. The data processing system can, therefore, simulate surgical steps directly on the 3D model, such as dissection, clamping, or suturing.

To provide guidance during the surgical simulation, the data processing system can provide the user with real-time feedback. The data processing system can provide real-time feedback on events such as damage to anatomical structures, whether any tumors are remaining or were left behind, the use of advance techniques such as selective clamping, instances of positive margins, or the percentage of the anatomical structure (e.g., kidney) that was spared (e.g., based on a calculation in real-time using the original kidney volume and the remaining kidney volume post dissection).

After the simulation, the data processing system can provide post simulation results. Post simulation results can include any of the real-time feedback on events. The data processing system can also show, post simulation, the dissected 3D model after the simulation for reference purposes. For example, the data processing system can show, via the graphical user interface, the 3D model of the kidney or organ with the tumor removed. The surgeon may refer to post simulation depiction of the kidney with the tumor removed for reference purposes. For example, the depiction can indicate whether the surgery was performed correctly or other performance metrics for the surgery. The data processing system can provide specific pictures or clips from the simulation that correspond to certain steps in the procedure, or events, that can be accessed during surgery. For example, in a partial nephrectomy, a kidney dissected in simulation can show the pathway taken by the surgeon for dissection and also the size or shape of the defect left by the surgeon after the tumor dissection. The data processing system can save the dissected 3D models post simulation for use in case reports or procedure review.

The data processing system can use the 3D models of the organ or anatomical structure to improve the digital simulation environment. For example, the incorporation of patient specific anatomical 3D models in simulation can lead to a more realistic simulation or visualization of anatomical structures. The data processing system can make the simulations more patient specific by adapting the simulated anatomy to match the 3D model. The data processing system can match the 3D model to the simulated anatomy using a model trained with machine learning that can take as input the 3D model in order to generate a simulated anatomy.

To create the 3D model of the anatomical structure, the data processing system can obtain a CT or MR imaging that represents the anatomical structure. The CT or MR imaging can represent a shape or volume of an organ, as well as the shape or volume of the a tumor within or on the organ. The data processing system can use these inputs from the CT or MR imaging to created a simulated kidney that matches the anatomical inputs.

The data processing system can compare the performance results obtain by a surgeon using the simulation with the performance results obtained by a surgeon when performing the actual surgery, and generate a report based on the comparison.

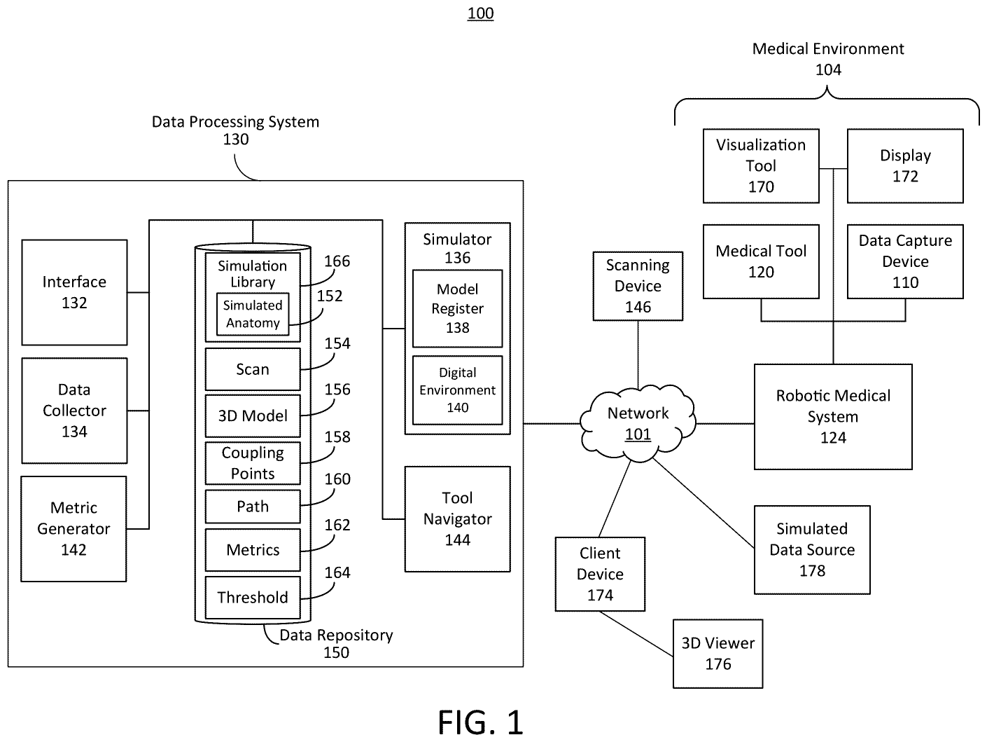

FIG. 1 depicts an example system 100 of tool navigation using a customized medical simulation, in accordance with implementations. The system 100 can be associated with a medical environment 104. The medical environment 104 can be a medical surgical environment. A medical surgical environment can include a surgical facility such as an operating room in which a surgical procedure, whether, invasive, non-invasive, in-patient, or out-patient, can be performed on a patient. The system 100 can be associated with other types of medical sessions or activities, or non-medical environments that can require removal of non-surgical information from a data stream captured from that environment. The system 100 can include one or more data capture devices 110. Data capture devices 110 can collect images, kinematics data, or system events, for example. For example, the data capture device can include an image capture devices designed, constructed and operational to capture images from a particular viewpoint within the medical environment 104. The data capture devices 110 can be positioned, mounted, or otherwise located to capture content from any viewpoint that facilitates the data processing system recognizing aspects of the procedure, such as phases of the procedure. The data capture devices 110 can include any of a variety of sensors, cameras, video imaging devices, infrared imaging devices, visible light imaging devices, intensity imaging devices (e.g., black, color, grayscale imaging devices, etc.), depth imaging devices (e.g., stereoscopic imaging devices, time-of-flight imaging devices, etc.), medical imaging devices (e.g., endoscopic imaging devices, or ultrasound imaging devices), or non-visible light imaging devices.

The images captured by the data capture devices 110 can be sent as a data stream component to a visualization tool 170 or the data processing system 130. A data stream component can be considered any sequence of digital encoded data or analog data from a data source such as the data capture devices 110. The visualization tool 170 can be configured to receive a plurality of data stream components and combine the plurality of data stream components into a single data stream.

The visualization tool 170 can receive a data stream component from a medical tool 120. The medical tool 120 can be any type and form of tool used for surgery, medical procedures or a tool in an operating room or environment associated with or having an image capture device. The medical tool 120 can be an endoscope for visualizing organs or tissues, for example, within a body of the patient. The medical tool 120 can include other or additional types of therapeutic or diagnostic medical imaging implements. The medical tool 120 can be configured to be installed in a robotic medical system 124.

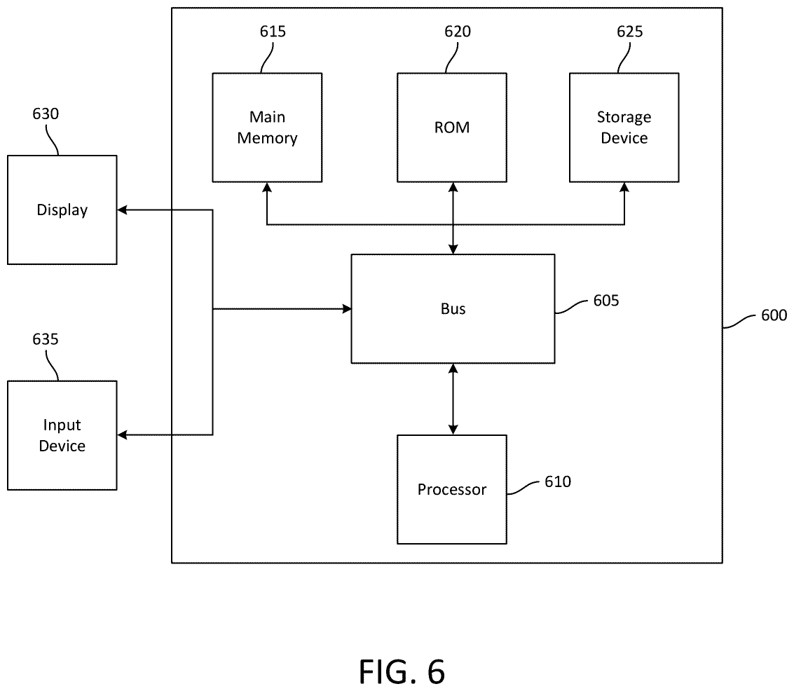

The robotic medical system 124 can be a computer-assisted system configured to perform a surgical or medical procedure or activity on a patient via or using or with the assistance of one or more robotic components or medical tools. The robotic medical system 124 can include one or more manipulator arms that perform one or more computer-assisted medical tasks. The medical tool 120 can be installed on a manipulator arm of the robotic medical system 124 to perform a surgical task. The images (e.g., video images) captured by the medical tool 120 can be sent to the visualization tool 170. The robotic medical system 124 can include one or more input ports to receive direct or indirect connection of one or more auxiliary devices. For example, the visualization tool 170 can be connected to the robotic medical system 124 to receive the images from the medical tool when the medical tool is installed in the robotic medical system (e.g., on a manipulator arm of the robotic medical system). The visualization tool 170 can combine the data stream components from the data capture devices 110 and the medical tool 120 into a single combined data stream for presenting on a display 172 (e.g., display 630 depicted in FIG. 6). The display 172 can be associated with a client device 174, user control system or other type of display system, whether within the medical environment 104 or remote, to view the single combined data stream. The client device 174 can refer to or include a laptop computer, desktop computer, tablet, smartphone, portable computing device, or wearable device, for example.

The system 100 can include a data processing system 130. The data processing system 130 can be associated with the medical environment 104, or cloud-based. The data processing system 130 can include an interface 132 designed, constructed and operational to communicate with one or more component of system 100 via network 101, including, for example, the robotic medical system 124 or client device 174. The data processing system 130 can include a data collector 134 to capture or otherwise receive or obtain data from one or more component or system associated with the medical environment 104 or via the network 101, including, for example, one or more data capture devices 110, a client device 174, or a scanning device 146. The data processing system 130 can include a simulator 136 to generate or establish a simulation of an anatomy of a subject. The simulator 136 can include a model register 138 to register or overlay a scan of an anatomical structure of the subject, such as an organ, onto the simulated anatomy of the subject. The simulator 136 can establish, generate, execute, or otherwise provide a digital environment 140 with which to provide an interactive simulation of the surgical procedure. The data processing system 130 can include a metric generator 142 to determine performance metrics associated with the execution of the simulation by the simulator 136, or performance metrics associated with the performance of the actual procedure in the medical environment 104. The data processing system 130 can include a tool navigator 144 to provide guidance before, during, or after the procedure based on the results of the executed by the simulator 136.

The interface 132, data collector 134, simulator 136, model register 138, digital environment 140, metric generator 142 or tool navigator can each communicate with the data repository 150 or database. The data processing system 130 can include or otherwise access the data repository 150. The data repository 150 can include one or more data files, data structures, arrays, values, or other information that facilitates operation of the data processing system 130. The data repository 150 can include one or more local or distributed databases, and can include a database management system.

The data repository 150 can include, maintain, or manage a simulation library 166. The simulation library 166 can include one or more types of simulated anatomies 152, simulated objects, simulation physics engines, digital environment attributes, or simulated medical tools. The simulated anatomy 152 can refer to, or include, a computer-based simulation to replicate structures or functions of an anatomy. The data processing system 130 can obtain the simulated anatomy 152 from a simulated data source 178, such as a third-party provider of simulated anatomies 152. The simulated anatomy 152 can be based on computer-generated models. The simulated anatomy 152 can include 3D models or animations that are static, in that they can have a default shape, size, or volume. In some cases, the simulated anatomy 152 can be parameterized such that parameters of the simulated anatomy 152 can be adjusted. For example, the data processing system 130 can adjust parameters such as the size, shape, or volume of the simulated anatomy 152. The simulated anatomy 152 can be stored in the data repository 150 as a mesh file, which can store the simulated anatomy 152 as a 3D model in a format such as a stereolithography (“STL”) or an OBJ format. The mesh file can represent the geometry of the simulated anatomy and can store information about the surfaces and structure of the simulated anatomy. The geometry can include information about points, lines or faces that allow the data processing system 130 (e.g., via simulator 136) to create 3D objects and the digital environment. In some cases, the simulated anatomy 152 can include texture maps or image files to enhance the visual appearance of the simulated anatomy. The texture maps or image files can add information such as color, texture, or other visual characteristics. In some cases, the simulated anatomy 152 can include simulation data, such as physics parameters, animation data, or interactive features. In some cases, the digital environment 140 can include such physics parameters, animation data or interactive features associated with the medical procedure to be simulated by the simulator 136. Examples of simulated anatomy 152 can include the body of a subject, organs, tissues, bones, vascular structures, or limbs.

The data repository 150 can include scan data 154. Scan data 154 can include medical imaging scans of an anatomical structure (e.g., an organ) of a subject or patient generated by a scanning device 146. The scan data 154 can include a scan of a tumor, growth, or other structure on or within the anatomical structure of the subject. The data processing system 130 can receive the scan data 154 from a scanning device 146. In some cases, the data processing system 130 can receive the scan data 154 via network 101. In some cases, the data processing system 130 can download or copy the scan data 154 from a storage medium, hard drive, or memory.

The scan data 154 can be generated using one or more medical imaging techniques. For example, the scan data 154 can be based on or include a computed tomography (“CT”) scan or a computerized axial tomography scan. A CT scan can be generated by a scanning device 146 that includes an X-ray source. The scanning device 146 can emit a beam of X-rays. The scanning device 146 can direct the beam of X-rays towards the anatomical structure in a subject or patient for which the medical procedure is to be performed via the robotic medical system 124 in the medical environment 104. In some cases, the scanning device 146 can be located in the medical environment 104, while in other cases the scanning device 146 can be located external or remote from the medical environment 104.

The scanning device 146 can include an X-ray detection system that captures the X-rays emitted by the scanning device 146 after the X-rays pass through the anatomical structure of the subject. The scanning device 146, or detector, can record the intensity of the captured X-rays. The scanning device 146 can include or be associated with a gantry system to cause the detector of the scanning device 146 to rotate around the anatomical structure, or subject thereof, to capture multiple X-ray projections at different angles. In some cases, the scanning device 146 can provide the captured X-ray data to the data processing system 130 for further processing. In some cases, the scanning device 146 can perform data processing on the captured X-ray data. For example, the scanning device 146 or data processing system 130 can use a filtered back projection technique to reconstruct a two-dimensional (“2D”) image for each cross-sectional slice of the captured X-ray data. The data processing system 130 or scanning device 146 can stack the reconstructed 2D images to generate a stack of slices that can create a 3D representation of the scanned anatomical structure.

In another example, the scanning device 146 can include a magnetic resonance imaging (“MRI”) machine. The MRI machine can generate a magnetic field, and apply bursts of radiofrequency pulses to the anatomical structure of the subject that is to be imaged. The scanning device 146 can detect radio frequency signals emitted by the anatomical structure through this process, and store the data. The scanning device 146 can store the data as a k-space data that represents the spatial frequencies of the signals. The data processing system 130 or scanning device 146 can process the k-space data and reconstruct an image using, for example, a Fourier transformation to convert the spatial frequency information into a spatial domain to create an image. The data processing system 130 or scanning device 146 can generate cross-sectional image of the anatomical structure. The data processing system 130 or scanning device 146 can generate 3D images from the 2D cross-sectional images (or slices) via image stacking, volumetric rendering, isosurface rendering, or multi-planar reconstruction, for example.

Thus, the scanning device 146 can obtain a patient-specific scan or image of the anatomical structure of the patient. The scan 154 can include an image of the organ (e.g., kidney), as well as any tumor, injury, infections, or abnormalities associated with the particular anatomical structure of the subject that is going to undergo the medical procedure in the medical environment 104 via the robotic medical system 124.

The data repository 150 can include a 3D model 156. The 3D model 156 can be reconstructed from the 2D image slices obtained by the scanning device 146. The slices can be stacked together to create a 3D representation of the scanned area. In some cases, segmentation can be performed to segment or separate out the anatomical structure of interest from other parts of the anatomy of the subject. For example, if a medical procedure is to be performed on a kidney, the data processing system 130 can segment out images or portions of images that include the kidney from the scan data 156 to generate a 3D model of the kidney. The 3D model of the kidney may exclude or other aspects of the anatomy that are not relevant. For example, the simulated anatomy 152 can include certain portions of anatomy that are not relevant to customize for a particular medical procedure. Thus, to reduce computing resource utilization, memory utilization, or network utilization, the data processing system 130 can generate a 3D model of the organ with a higher resolution, and overlay the 3D model of the organ with the higher resolution onto the simulated anatomy 152 that may be in lower resolution. In some cases, the 3D model 156 can be simulated, at least in part, by the simulator 136 using a model trained with machine learning and scan data 154 for the subject.

The 3D model 156 can be stored in various file format, including, for example, a gLTF/GLB file, an .OBJ file (e.g., wavefront object), a .USDZ/USD file, a PLY file (e.g., polygon file format), or other file format. The 3D model 156 can include information about a geometry (e.g., shape of the anatomical structure), appearance (e.g., color), scene (e.g., position of structures in the anatomy), or animations (e.g., movement of objects or structures in the anatomy). The file format can include a text format, comma separated files, data structures, or other formats.

The data repository 150 can include one or more coupling points 158. The coupling points 158 can include static points associated with the simulated anatomy 152 and the 3D model 156 of the anatomical structure. The data processing system 130 can use the coupling points 158 to register or overlay the patient-specific 3D model 156 onto the simulated anatomy 152. Coupling points 158 can refer to or include any points that facilitate the model register 138 to register, overlay, couple, or otherwise connect the 3D model 156 with simulated anatomy 152. Coupling points 158 can include, for example, veins, vascular structures, arteries, nerves, or other interfacing structures.

The data repository 150 can include a path 160. The path 160 can refer to or include a path generated via the simulator 136 during an executed simulation. The path 160 can refer to a path for a medical tool 120, such as a cutting tool, to perform a task in a medical procedure, such as a dissection. The data repository 150 can store multiple paths or candidate paths. The path 160 can include coordinates, vectors, lines, or points. The path 160 can use coordinates or points in a reference frame relative to, or established by, the robotic medical system 124. The reference frame can correspond to a the 3D model 156 or simulated anatomy 152. The reference frame can be established by the simulator 136 for the digital environment 140. The data repository 150 can include or store metrics 162 associated with the paths. The metrics 162 can refer to performance metrics, such as duration, efficiency, or outcome. The metrics 162 can be generated by the metric generator 142. The path 160 and metrics 162 can be associated with a particular task or phase of the medical procedure. For example, each task or phase of the medical procedure can have a separate path 160 and corresponding metrics 162. The data processing system 130 can use different paths for different phases of the medical procedure to optimize the overall performance of the medical procedure.

The data repository 150 can include, store, manage, or otherwise maintain a threshold data structure 164 used to facilitate tool navigation via customized 3D models, such as threshold for performance metrics. The threshold 164 can refer to or include a numerical value that can be used to determine whether a metric indicative of performance of a candidate path is satisfactory. For example, a metric for efficiency can be a length of a path, duration the tool 120 takes to traverse the path, whether any injury or damage was caused to the organ using the path, whether the tumor was fully removed, percentage of removal, instances of positive margins, or the percentage of the anatomical structure (e.g., kidney) that was spared (e.g., based on a calculation in real-time using the original kidney volume and the remaining kidney volume post dissection). The value for the metric for a medical procedure can be compared with the threshold for the metric to determine whether the value for the metric exceeds the threshold for the metric, in which case the data processing system 130 can determine that the layout is inefficient and perform an action to cause a change in the layout.

In some cases, the threshold data structure 164 can include or refer to a map of thresholds. The thresholds can map to one or more attributes, factors, or categories associated with a medical environment 104 or medical procedure. For example, the threshold can map to an institution in which the medical procedure is to be performed, a type of medical procedure, or a phase in the medical procedure. To evaluate a performance of a type of the medical procedure being simulated or performed in the medical environment 104, the data processing system 130 can select the corresponding threshold via a lookup in the threshold data structure 164 using the type of medical procedure, surgeon identifier, or an attribute of the 3D model of the anatomical structure.

The data repository 150 can store, maintain, or manage other or additional information that facilitates tool navigation using a customized medical simulation. For example, the data repository 150 can store a data stream received by the data collector. The data stream can include or be formed from one or more of a video stream, event stream, or kinematics stream. The data stream can include data collected by one or more data capture devices 110.

The event stream can include a stream of event data or information, such as packets, that identify or convey a state of the robotic medical system 124 or an event that occurred in association with the robotic medical system 124 or surgical or medical surgery being performed with the robotic medical system. Data of the event stream can be captured by the robotic medical system 124 or a data capture device 110. An example state of the robotic medical system 124 can indicate whether the medical tool 120 is installed on a manipulator arm of the robotic medical system or not, whether it was calibrated, or whether it was fully functional (e.g., without errors) during the procedure. For example, when the medical tool 120 is installed on a manipulator arm of the robotic medical system 124, a signal or data packet(s) can be generated indicating that the medical tool has been installed on the manipulator arm of the robotic medical system 124. The signal or data packet(s) can be sent to the data collector 134 as the event stream. Another example state of the robotic medical system 124 can indicate whether the visualization tool 170 is connected, whether directly to the robotic medical system or indirectly through another auxiliary system that is connected to the robotic medical system.

Kinematics stream data can refer to or include data associated with one or more of the manipulator arms or medical tools 120 attached to manipulator arms, which can be captured or detected by one or more displacement transducers, orientational sensors, positional sensors, or other types of sensors and devices to measure parameters or generate kinematics information. The kinematics data can include sensor data along with time stamps and an indication of the medical tool 120 or type of medical tool 120 associated with the sensor data.

The data repository 150 can include, store, manage, or otherwise maintain phases data. The phases can refer to or include operative phases or non-operative phases (e.g., operating room related phases), such as room preparation, robot setup, performance of a medical procedure, turn over, or cleaning, for example. In some cases, phases can refer to or include operative phases, such as exposure, dissection, transection, reconstruction, and extraction. Exposure can refer to or include the process of visualizing and accessing a surgical site by creating a clear and adequate field of view. Dissection can refer to or include cutting, separating and removing tissues or anatomical structures to gain access to specific areas, identify structures, or perform surgical procedures. Transection can refer to or include severing or cutting a structure, such as a blood vessel, nerve, or organ using a surgical instrument. Extraction can refer to or include the removal of a tissue, organ, foreign object, or other anatomical structure from the body. Reconstruction can refer to or include the process of restoring or rebuilding a damaged or missing tissue, organ, or body part, and can include techniques or tasks such as grafting, suturing, or using prosthetic materials to recreate the structure and restore form and function.

The data repository 150 can include, manage or maintain historical data. Historical data can include prior video stream, event stream, or kinematic stream data.

The data processing system 130 can interface with, communicate with, or otherwise receive or provide information with one or more component of system 100 via network 101, including, for example, the robotic medical system 124 or client device 174. The data processing system 130, robotic medical system 124 or client device 174 can each include at least one logic device such as a computing device having a processor to communicate via the network 101. The data processing system 130, robotic medical system 124 or client device 174 can include at least one computation resource, server, processor or memory. For example, the data processing system 130 can include a plurality of computation resources or processors coupled with memory.

The data processing system 130 can be part of or include a cloud computing environment. The data processing system 130 can include multiple, logically-grouped servers and facilitate distributed computing techniques. The logical group of servers may be referred to as a data center, server farm or a machine farm. The servers can also be geographically dispersed. A data center or machine farm may be administered as a single entity, or the machine farm can include a plurality of machine farms. The servers within each machine farm can be heterogeneous—one or more of the servers or machines can operate according to one or more type of operating system platform.

The data processing system 130, or components thereof can include a physical or virtual computer system operatively coupled, or associated with, the medical environment 104. In some embodiments, the data processing system 130, or components thereof can be coupled, or associated with, the medical environment 104 via a network 101, either directly or directly through an intermediate computing device or system. The network 101 can be any type or form of network. The geographical scope of the network can vary widely and can include a body area network (BAN), a personal area network (PAN), a local-area network (LAN) (e.g., Intranet), a metropolitan area network (MAN), a wide area network (WAN), or the Internet. The topology of the network 101 can assume any form such as point-to-point, bus, star, ring, mesh, tree, etc. The network 101 can utilize different techniques and layers or stacks of protocols, including, for example, the Ethernet protocol, the internet protocol suite (TCP/IP), the ATM (Asynchronous Transfer Mode) technique, the SONET (Synchronous Optical Networking) protocol, the SDH (Synchronous Digital Hierarchy) protocol, etc. The TCP/IP internet protocol suite can include application layer, transport layer, internet layer (including, e.g., IPv6), or the link layer. The network 101 can be a type of a broadcast network, a telecommunications network, a data communication network, a computer network, a Bluetooth network, or other types of wired and wireless networks.

The data processing system 130, or components thereof, can be located at least partially at the location of the surgical facility associated with the medical environment 104 or remotely therefrom. Elements of the data processing system 130, or components thereof can be accessible via portable devices such as laptops, mobile devices, wearable smart devices, etc. The data processing system 130, the data collector 134, or components thereof, can include other or additional elements that can be considered desirable to have in performing the functions described herein. The data processing system 130, or components thereof, can include, or be associated with, one or more components or functionality of computing system 600 depicted in FIG. 6, including, for example, one or more processors coupled with memory.

The data processing system 130 can include an interface 132 designed, constructed and operational to communicate with one or more component of system 100 via network 101, including, for example, the robotic medical system 124 or client device 174. The interface 132 can include a network interface. The interface 132 can include or provide a user interface, such as a graphical user interface.

The interface 132 can provide data for presentation via a 3D viewer 176 that can depict, illustrate, render, present, or otherwise provide a digital environment 140 provided by the data processing system 130 (e.g., simulator 136). The interface 132 can provide the 3D viewer 176 application via a web browser or native application executing on the client device 174. The 3D viewer 176 can be a software-as-a-service application hosted by the data processing system 130 or one or more servers. The 3D viewer 176 can be a native application hosted or executed on a client device 174.

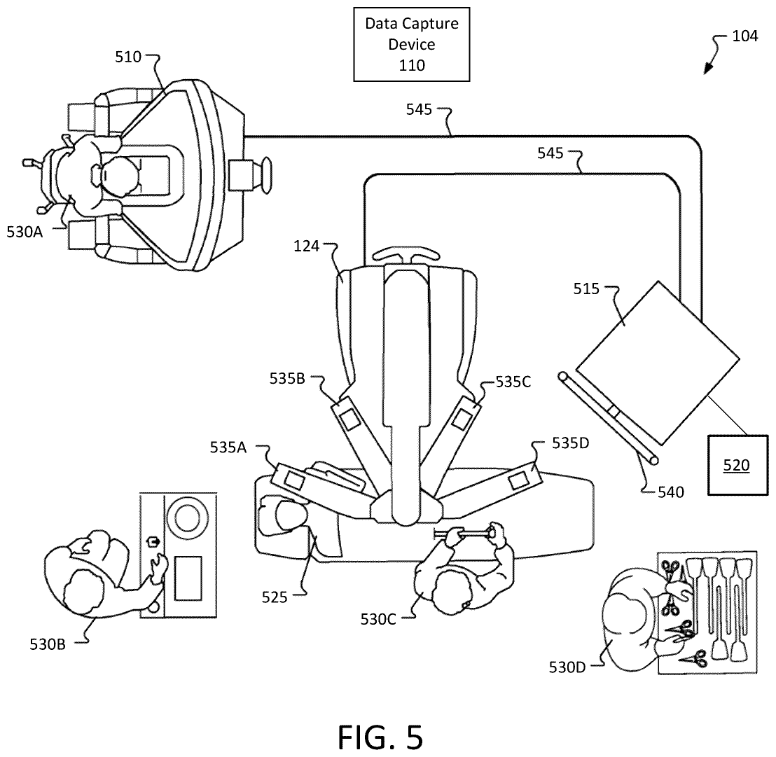

The data processing system 130 can include a data collector 134 designed, constructed and operational to receive sensor data associated with a medical procedure and captured by a set of sensors (e.g., multiple data capture devices 110) situated in an operating room (e.g., medical environment 104 depicted in FIG. 5). The data collector 134 can receive scanned data 154 from the scanning device 146. The data collector 134 can obtain simulated data from various sources via network 101, such as a simulated data source 178.

The data processing system 130 can include a simulator 136 designed, constructed and operational to simulate a medical procedure. The simulator 136 receives information about a type of medical procedure to simulate. For example, the simulator 136 can receive one or more of a type of medical procedure to simulate, an identifier of a patient or subject for which to simulate the medical procedure, types of instruments or robotic medical systems 124 to use to simulate the medical procedure, or phases of the medical procedure to simulate. The data processing system 130 can receive the information or instructions via the interface 132, such as input device 635.

The simulator 136 can generate various types of simulations. The simulator 136 can generate a simulation using a standard, default, or static simulated anatomy 152 received from a third-party simulated data source 178. However, the simulated anatomy 152 received from the third-party data source 178 may not be customized for a particular subject on which a medical procedure is to be formed. For example, the organ may have a different shape or size, or a tumor that is to be removed from the organ may have a different shape or size, relative to the shape or size established in the default, static simulated anatomy. By executing a simulation of an anatomical structure that is not representative or sufficiently customized to the actual medical procedure to be performed, the robotic medical system 124 may be configured with an erroneous or inefficient navigation path for the medical tool 120, or provide erroneous or inefficient guidance to navigate the medical tool 120.

Thus, aspects of this technical solution include a simulator 136 with a model register 138 that is designed, constructed and operational to access a 3D model 156 of an anatomical structure (e.g., a kidney with a tumor) generated via a scan 154 of a subject on which a procedure is to be performed via the robotic medical system 124. The model register 138 can select the 3D model 156 from multiple 3D models 156 stored in the data repository 150. The model register 138 can select the 3D model 156 based on performing a lookup in the data repository 150 using an identifier associated with the subject on which the medical procedure is to be performed. In some cases, the model register 138 can select the 3D model 156 based on a procedure identifier, timestamp, date, location, or other criteria. The data processing system 130 can provide a graphical user interface that depicts the various 3D models 156 available for a particular subject, from which a user of the data processing system 130 can select a 3D model 156 to register in the digital environment 140. In some cases, the data processing system 130 can filter the available 3D models 156 based on the type of procedure to be performed. For example, if the type of procedure to be performed is a partial nephrectomy, then the data processing system 130 can either automatically select a 3D model 156 that corresponds to the kidney organ type, or, if there are multiple 3D models 156 for the subject identifier that match the kidney organ type, then provide a list of the available kidney 3D models 156. In some cases, the data processing system 130 can automatically select the most recently generated 3D model 156 for matching procedure type.

In some cases, the model register 138 or simulator 136 can generate a customized 3D model 156 from scan data 154 using a model trained with machine learning. The simulator 136 can input the scan data 154 into a machine learning model in order to generate a 3D model 156 that is customized for the specific subject of the medical procedure based on the scan data 154 received via scanning device 146. The data processing system 130 can use a series of images in the scan data 154, such as a stack of image slices, to perform feature extraction. The data processing system 130 can identify and extract distinctive features in each image of the scan data 154. The features can act as reference points for matching between images. Example feature extraction techniques can include scale-invariant feature transform (“SIFT”) or speeded-up robust features (“SURF”). The model register 138 can use the feature extraction to identify relationships between the different images, and then create 3D coordinates of each point based on a reference frame. The data processing system 130 can combined the 3D coordinates of matched points to create a point cloud, which can include a set of 3D points representing the anatomical structure. The data processing system 130 can create a surface mesh from the point cloud and perform texture mapping based on the type of anatomical structure or attributes of the anatomical structure, to result in the generation of the 3D model 156. The data processing system 130 can save this 3D model 156 in the data repository 150 for further processing or use. The 3D model can be stored in the OBJ or STL file format, for example.

Upon accessing the customized 3D model 156 from the data repository 150, the model register 138 can register the 3D model 156 of the anatomical structure with predetermined coupling points 158 in the digital environment 140 established for the procedure on the subject. Registering the 3D model 156 in the digital environment 140 can refer to or include overlaying the customized 3D model 156 on the simulated anatomy 152 in the digital environment 140. Registering the 3D model 156 can include resizing the simulated anatomy 152 to match the 3D model 156, scaling one or both of the 3D model 156 or the simulated anatomy 152, re-orienting one or more both the 3D model 156 or the simulated anatomy 152, or otherwise aligning the 3D model 156 with the corresponding portion of the simulated anatomy 152.

For example, the simulated anatomy 152 can include one or more anatomical structures that are adjacent to, surround, or otherwise proximate to the anatomical structure that is to be replaced by the customized 3D model 156. The model register 138 can identify couple points 158 in the simulated anatomy 152. The coupling points 158 can include an identifier, label, or other indication. The 3D model 156 can include a corresponding identifier, label or other indication. The model register 138 can identify the coupling points 158 on the simulated anatomy 152 and the corresponding coupling points 158 on the 3D model 156. The model register 138 can connect the coupling points 158 on the simulated anatomy 152 with the corresponding coupling points on the 3D model 156 in order to register the 3D model 156 with the simulated anatomy 152.

For example, the couple points 158 can include a first vein and a second vein. The first vein can refer to or include an input vein that caries blood from an anatomical structure in the simulated anatomy 152 into the 3D model 156 of the organ. The second vein can include an output vein that carries blood out of the 3D model 156 of the organ and back into the simulated anatomy 152. To register the 3D model 156 with the simulated anatomy 152, the model register 138 can align a first point (e.g., a first couple point 158) on the 3D model 156 of the organ with the first vein in the simulated anatomy 152. The model register 138 can align a second point (e.g., a second coupling point 158) on the 3D model 156 of the organ with the second vein in the simulated anatomy 152. The simulated anatomy 152 can be a default, or standard representation of the subject that may not be customized for the subject, whereas the 3D model 156 can be customized for the organ of the subject and generated based on the scan data 154.

The model register 138 can adjust a size of the simulated anatomy 152 to register the 3D model 156 of the organ with the simulated anatomy 152 in the digital environment 140. For example, the model register 138 can determine that the size of the anatomical structure in the customized 3D model 156. The model register 138 can determine the size based on the couple points 158 of the 3D model 156. The model register 138 can determine the volume of the 3D model 156. The model register 138 can determine, based on the coordinates or locations of the couple points of the 3D model 156 and the coupling points on the simulated anatomy 152, that there is a mismatch that prevents the 3D model 156 from being registered or overlayed accurately onto the simulated anatomy 152. To correct, address, or other remedy this mismatch, the model register 138 can re-size the 3D model 156 or the simulated anatomy 152. In some cases, the model register 138 can determine to maintain the same size of the 3D model 156 because the 3D model 156 is customized for the particular subject that is going to undergo the medical procedure. As such, the model register 138 can determine to re-size the simulated anatomy 152 such that the coupling points 158 of the 3D model 156 connect, link, couple, or otherwise align with the coupling points 158 on the simulated anatomy.

The model register 138 can adjust an orientation of the 3D model 156 or the simulated anatomy 152 to register or overlay the 3D model 156 with the simulated anatomy 152. The model register 138 can adjust the orientation of the 3D model 156 to align the coupling points 158 of the 3D model with the corresponding coupling points of the simulated anatomy 152. Adjusting an orientation can refer to or include adjusting a yaw, pitch or roll of the 3D model 156 or the simulated anatomy 152 relative to a reference frame. The reference frame can be established based on the simulated anatomy 152, or some other reference frame in the digital environment 140.

The model register 138, to improve computing efficiencies associated with executing or rendering a digital simulation of the medical procedure in the digital environment 140, can select different resolutions for different aspects or portions of the simulation. For example, the model register 138 can select a higher resolution 3D model 156 of the organ on which the medical procedure is performed, while selecting a lower resolution simulated anatomy 152 for portions that are adjacent to the organ and may not be directly interacted with during the medical procedure. To do so, the simulator 136 can receive an indication of the type of procedure to be performed or the portions of the subject ton which the procedure is to be performed. For example, if the procedure type is a nephrectomy, then the simulator 136 can determine to select a higher resolution 3D model 156 of the kidney, while selecting a lower resolution simulated anatomy 152 of anatomical structures that are adjacent to the kidney. In some cases, a user of the data processing system 130 can highlight a region of the simulated anatomy 152 where the medical procedure is to be performed, and the simulator 136 can, responsive to the indication of the highlighted region, select a higher resolution 3D model 156 for anatomical structures within the region.

Resolution can refer to an image or image stack with a spatial resolution, texture resolution, color depth, dynamic resolution, or multi-scale representation. A high spatial resolution can represent tissues or anatomical structures measured in millimeters or even smaller. For example, a high spatial resolution can resolve anatomical structures on the scale of 10 millimeters, 9 millimeters, 8 millimeters, 5 millimeters, 3 millimeters, or smaller. A low spatial resolution can refer to resolving anatomical structures on the scale of 20 millimeters, 25 millimeters, 30 millimeters, 30 millimeters, 50 millimeters, 1 centimeter, or bigger. Texture resolution can refer to representing surface characteristics of organs, tissues, or other structures. High texture resolution can be 2,000 (2K) pixels (e.g., 2048×1080 pixels) or 4,000 (4K) pixels (e.g., 3840×2160 pixels) or higher. Low texture resolution can be lower, such as 1K. Color depth can refer to the level of accuracy with which the variety of colors found in anatomical structures are represented in the digital environment 140. For example, a high color depth can be 32-bit color, 128-bit color, 256-bit color, or more. Low color depth can be 16-bit color, 8-bit color, or lower. Dynamic resolution can refer to the ability to update or depict changes in real-time with a high level of accuracy. For example, high dynamic resolution can refer to updating at a high rate of 60 frames per second or more, while low dynamic resolution can refer to updating at a lower rate, such as 24 frames per second or lower.

Thus, depending on the type of medical procedure or the anatomical structure on which the medical procedure is to be performed, the medical register 138 can select a resolution for the 3D model 156 and a different resolution for the simulated anatomy 152, and then register the 3D model 156 with the simulated anatomy 152. The model register 138 an perform a lookup based on the type of medical procedure, organ, or preferences of the user to identify the resolutions. In some cases, the model register can provide the available resolution options to the user, and receive a selection of the resolution. In some cases, the simulator 136 can suggest resolutions or automatically select resolutions based on the hardware configuration, computing capacity, or network capacity that is available to execute the simulation.

The simulator 136 can execute a simulation in the digital environment 140. The digital environment 140 can include a computer-generated or virtual space in which the simulation takes place. The simulator 136 can create the digital environment 140 using computer graphics to replicate real-world medical scenarios or systems. The digital environment 140 can simulate physics using a physics engine that is configured to simulate the behavior of anatomical structures, medical tools 120 or other objects, materials or forces. The digital environment 140, using the physics engine, can facilitate realistic interactions and responses within the simulated space.

The digital environment 140 can refer to or include a simulation of at least a portion of the medical environment 104. The digital environment 140 can include the 3D model 156 registered or overlayed on the simulated anatomy 152. The digital environment 140 can be interactive. The digital environment 140 can mimic, emulated, or otherwise simulate the medical environment 104 or performing a medical procedure using the robotic medical system 124. The simulator 136 can present the digital environment 140 via the 3D viewer 176 of the client device 174 in XR.

The data processing system 130 can monitor or track the performance of the simulated medical procedure. The user of the data processing system 130 or surgeon can practice performing the medical procedure in the simulated digital environment 140. The user can interact with the digital environment 140 to simulate the medical procedure using the client device 174 or a user control system 510. For example, the data processing system 130 can render or provide the digital environment 140 for display via the user control system 510.

The data processing system 130 can track user inputs to collect data. The data can include any type of data captured by the data collector 134. The type of data collected during the simulation can include one or more types of data captured in a data stream by the robotic medical system 124, such as a video stream, kinematics stream, or event stream. The data can include information about a type of the medical tool 120 used in the simulation, an angle or orientation of the simulated medical tool 120, or angle of approach relative to the 3D model 156 of the organ. The data can include movements, gestures, or actuations of the simulated medical tool 120. The data can include duration or efficiency information.

The data processing system 130 can simulate performance data or results of the simulated medical procedure. The data processing system 130 can include a metric generator 142 designed, constructed, and operational to determine the performance data. The performance data can refer to or include values for performance metrics. The metric generator 142 can use the simulated data to generate various types of objective performance indicators, metrics or otherwise provide feedback on events associated with the simulated medical procedure. The metric generator 142 can store the metrics or feedback on events in the metric data structure 162. The metric generator 142 can associate values of the metrics or the feedback on the events with time stamps during the simulated medical procedure. For example, the metric data structure 162 can store, for a particular simulation, a time series of metrics or a time series of feedback events in the metric data structure 162.

Example performance metrics can include the duration of the medical procedure (or a phase thereof), amount of energy consumed during the medical procedure (or phase thereof), instances of positive margins (e.g., the presence of cancer cells at the edge of the tissue or organ that was removed during surgery), or the percentage of the anatomical structure (e.g., kidney) that was spared (e.g., based on a calculation in real-time using the original kidney volume and the remaining kidney volume post dissection). The data processing system 130 (e.g., via metric generator 142) can provide real-time feedback on events such as damage to anatomical structures, whether any tumors are remaining or were left behind, or when any advance techniques, such as selective clamping, were used during the simulation.

The data processing system 130 (e.g., via metric generator 142) can determine a performance metric that indicates the percentage remaining of an organ after the medical procedure. For example, the data processing system 130 can determine the amount of the simulated organ that was cut or removed during the simulation. The amount can refer to or include an absolute amount, such as a volume or weight. The amount can refer to or include a percentage or ratio that is remaining relative to the original, starting volume of the 3D model 156 of the anatomical structure. The amount can refer to or include a percentage or ratio that is removed relative to the original, starting volume of the 3D model 156 of the anatomical structure.

To do so, and for example, the data processing system 130 can compute, using the 3D point cloud corresponding to the 3D model and the surface mesh information, the interactions between the simulated medical tool and the registered 3D model 156. The data processing system 130 can compute an original volume or shape of the 3D model 156, and then compute a remaining volume of the 3D model 156 after the simulated medical procedure is complete. The data processing system 130 can divide the remaining volume by the original volume to determine the ratio or percentage remaining of the anatomical structure.

The data processing system 130 (e.g., the metric generator 142 or simulator 136) can track the path of the simulated during the simulation of the medical procedure. The data processing system 130 can track the path using coordinates, vectors, location points, or any other technique. The data processing system 130 can associate the points or coordinates in the path with times stamps during the simulated medical procedure. The data processing system 130 can store the path in the path data structure 160. The data processing system 130 can associate or link the path data structure 160 with the metrics data structure 162. The data processing system 130 can generate performance metrics for each path of the medical tool generated in the simulation.

After the surgeon or user complete the medical procedure in the simulated digital environment using the customized 3D model 156 registered with the simulated anatomy 152, the data processing system 130 can provide the performance metrics 162 for the path generated during the simulation. The metric generator 142 can compare the performance metrics 162 with a threshold 164. The metric generator 142 can display the performance metrics via a graphical user interface. The metric generator 142 can provide a resulting values for performance metrics. The values for the performance metrics can be a numeric value, a percentage, a binary value (e.g., good or bad, positive margin or negative margin), a score, or a letter grade. The metric generator 142 can compare a numeric value of a performance metric with a threshold 164, and provide the results of the comparison. In some cases, the metric generator 142 can normalize a value of the performance metric based on a threshold 164.

The data processing system 130 can allow the surgeon to perform multiple simulations of the medical procedure. The data processing system 130 can vary aspects of the digital environment 140, or maintain a static digital environment 140 for each iteration of the simulation. For example, to the extent there may be parameters of the digital environment that can vary, the simulator 136 can apply a Monte Carlo technique to vary the values of the parameters based on a function, a probability range, or randomly. The data processing system 130 can generate performance metrics or feedback on events for each iteration of simulated medical procedure. The data processing system 130 can record a track of the simulated medical tool 120 for each simulation of the medical procedure, and store the path in the path data structure 160 and store the generated performance metrics in the metrics data structure 162. Thus, the data processing system 130, for a given medical procedure, can generate multiple simulated paths and performance metrics for storage in the data processing system 130. The metric generator 142 can generate an overall score for each simulated path that is based on multiple performance metrics. For example, the metric generator 142 can input values for each of multiple performance scores into a function (e.g., an average, weighted average, or other function) to determine an overall score for the path. The metric generator 142 can rank the simulated paths based on the overall score for each simulated path.

In some cases, if values of the performance metric do not satisfy a threshold for the performance metric, the data processing system 130 can generate a notification, indication, alert, or guidance to re-simulate the medical procedure in order to satisfy the threshold. For example, if the percentage of the organ that remains after the simulated medical procedure is below a threshold that would render the organ non-functional (e.g., 40%), then the data processing system 130 can provide a visual alert via the graphical user interface or an auditory alert to indicate the unsatisfactory result, and then the data processing system 130 can automatically initialize the digital environment 140 and re-execute the digital environment 140 with the initial conditions (e.g., access the customized 3D model 156 of the organ and register the organ with the simulated anatomy 152). In some cases, the data processing system 130 can continue to iterate through the simulations until the threshold 164 has been satisfied. In some cases, the data processing system 130 can provide suggestions to improve the value of the performance metric. In some cases, the data processing system 130 can overlay the paths generated from each simulation together with a time series of values of performance metrics for each path to illustrate when or where each path satisfied or failed the threshold. Thus, the data processing system 130 can provide guidance as to how to improve the performance of the medical procedure using the customized 3D model of the anatomical structure on which the medical procedure is to be performed.

The data processing system 130 can include a tool navigator 144 designed, constructed and operational to provide, for display via a graphical user interface (e.g., the 3D viewer 176), an indication of a candidate path for the medical tool 120 to perform the procedure via the robotic medical system 124. The tool navigator 144 can leverage the metrics data structure 162 and the paths data structure 160 to provide a candidate path for a medical procedure. The tool navigator 144 can depict the simulated paths via a graphical user interface prior to a medical procedure, during a medical procedure, or after the completion of the medical procedure. The tool navigator 144 provide the simulated path for pre-operation planning, intra-operative reference, or post-operative case review, for example. The tool navigator 144 can provided optimized guidance based on the customized 3D model of the anatomical structure for a particular task, phase, or other case level of a medical procedure. The tool navigator 144 can automatically recommend or suggest a best or optimized candidate path from multiple available candidate paths (e.g., in path data structure 160) based on objective performance indicators (e.g., metrics 162) for the surgeon to use during the actual medical procedure. The data processing system 130 can display the various candidate paths, along with corresponding performance metrics, and highlight optimal or highest scoring paths (e.g. use different colors, line thickness or other visual indicators).

For example, the tool navigator 144 can identify multiple candidate paths that were generated during multiple simulations for a particular medical procedure that is to be performed on a subject. The tool navigator 144 can identify the candidate paths responsive to a request from a user, or responsive to detecting the occurrence or initiation of a pre-operative phase for the medical procedure. The tool navigator 144 can identify a first value for a performance metric for a first candidate path of the multiple candidate paths, and identify a second value for the performance metric for a second candidate path of the multiple candidate paths. For example, the tool navigator 144 can perform a lookup in the metrics data structure 162 using an identifier for the candidate path to identify the value for the metric. In some cases, the tool navigator 144 can request the metric generator 142 to compute the value for the performance metric. The metric generator 142, responsive to the request from the tool navigator 144, can compute the metric using the path data structure 160 or other data recorded for the simulation that generated the path. The tool navigator 144 can compare the first value of the performance metric with the second value of the performance metric. The tool navigator 144 can select the candidate path based on a comparison of the first value and the second value. The tool navigator 144 can provide, responsive to the selection, the indication for the candidate path for display via the graphical user interface. The data processing system 130 can display one or more values of the performance metric in association with one or more candidate paths. The interface 132 or client device 174 can provide a graphical user interface on which to display the values for the performance metrics.

For example, the performance metric can be the duration of the medical procedure. The tool navigator 144 can determine that both the first and second paths were viable paths in that they resulted in the medical procedure having a satisfactory outcome. Tool navigator 144 can determine that since both paths resulted in satisfactory outcomes, that the optimal path corresponds to the path with the shorter duration. Thus, the tool navigator 144 can select the candidate path having the shorter value for the duration performance metric.

The tool navigator 144 can compare one or more performance metrics among the candidate paths or with thresholds to rank the candidate paths and provide a suggestion of a top ranking candidate path. The data processing system 130 can select the threshold based on the type of medical procedure or a type of organ associated with the medical procedure. For example, the data processing system 130 can identify a value of a performance metric related to the candidate path for the tool after the execution of the simulation of the medical procedure has completed. The data processing system 130 can identify a threshold for the performance metric based on at least one of a type of procedure or a type of the organ. The data processing system 130 can compare the value for the performance metric with the selected threshold. The data processing system 130 can provide, responsive to the value of the performance metric satisfying the threshold, the candidate path for display via the graphical user interface. The data processing system 130 can receive an indication via the interface 132 to select the candidate path for utilization during the medical procedure.

The data processing system 130 can overlay, via the graphical user interface, the indication of the candidate path on the 3D model 156 of the organ. The data processing system 130 can overlay the selected or suggested candidate path. The data processing system 130 can receive a selection of the candidate path via interface 132, and then overlay the candidate path on the 3D model of the organ responsive to the selection. The data processing system 130 can overlay the selected candidate path to facilitate performance of the medical procedure.

In some cases, the data processing system 130 the data processing system 130 can display the candidate path, and the user or surgeon can reject the candidate path. For example, the surgeon can determine that a value of a performance metric is not satisfactory, even though it may satisfy a threshold. In another example, the surgeon can determine that a particular path appears to be risky or unnecessarily complicated, even though the path may be associated with satisfactory values of performance metric. The surgeon may determine that a second candidate path, while having a lower value of a performance metric, may be less risky or more conservative. Thus, the data processing system 130 can receive, subsequent to the display of the candidate path, a command to reject the candidate path displayed via the graphical user interface. The data processing system 130 can remove, responsive to the command to reject the originally selected or displayed candidate path, the display of the candidate path. The data processing system 130 can then display a second path identified during execution of the simulation responsive to the request to reject the first candidate path. For example, the data processing system 130 can perform a lookup in the paths data structure 160 to identify a next highest ranking candidate path, and display the next highest ranking candidate path.

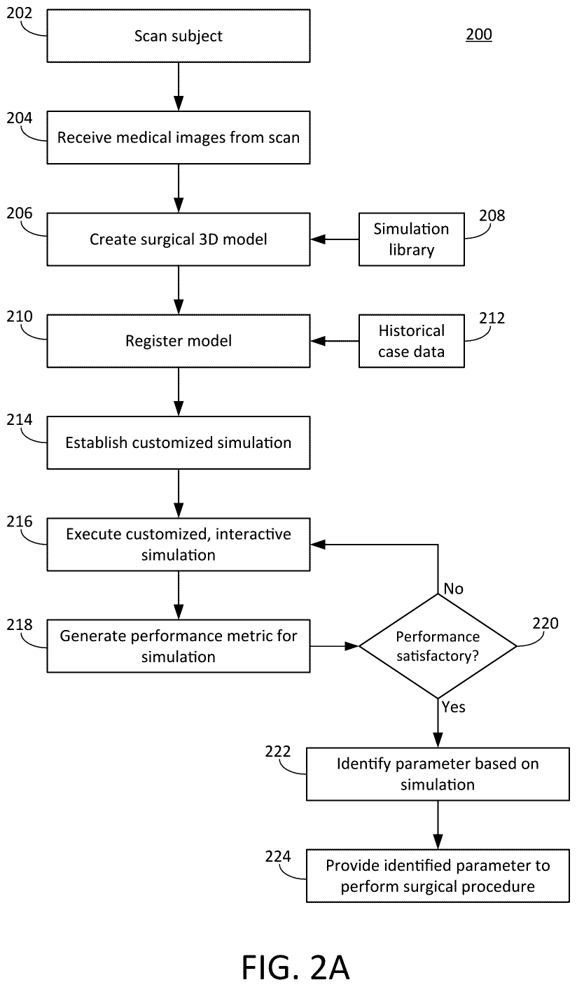

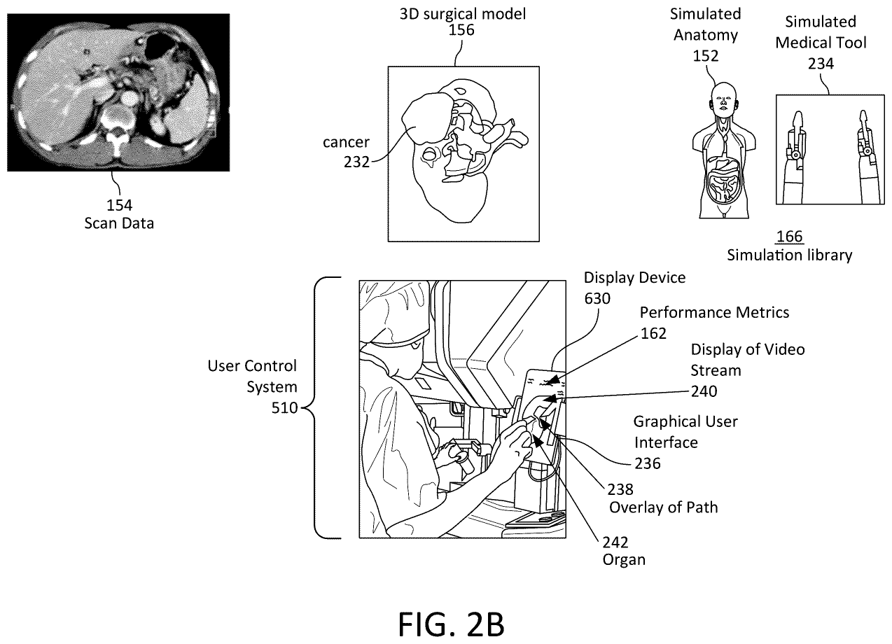

To facilitate the performance of the medical procedure, the tool navigator 144 can display the simulated path with a live video stream of the anatomical structure taken by a data capture device 110 in the medical environment 104. For example, the data processing system 130 can receive a video stream captured by a camera of the procedure performed via the robotic medical system 124 on the subject in a medical environment. The data processing system 130 can display the video stream via the graphical user interface (e.g., graphical user interface 236 depicted in FIG. 2B). The data processing system 130 can overlay the indication of the candidate path (e.g., overlay 238 depicted in FIG. 2B) for the tool through the organ (e.g., organ 242 depicted in FIG. 2B) on the video stream of the procedure displayed via the graphical user interface (e.g., display of video stream 240 depicted in FIG. 2B). The overlay of the path can be displayed using different visual attributes, such as a different color, transparency level, or dotted line. The surgeon, during the medical procedure, can use the path overlayed on the real-time video stream of the organ 242 to facilitate performance of the medical procedure on the organ. For example, the surgeon can trace the simulated path in order to reproduce the same or similar performance outcome during the simulation.

The data processing system 130 can receive a data stream captured by the data capture device 110 of the actual medical procedure, and generate values for performance metrics based on the data stream. The data processing system 130 can generate values for one or more of the same performance metrics for which the metric generator 142 generate values during the simulation. The data processing system 130 can compare the values of the same performance metrics for the actual medical procedure with the values of the performance metrics generated during the simulation that generated the simulated path that was used to facilitate the performance of the medical procedure. The data processing system 130 can generate the values of the performance metrics in real-time (e.g., during performance of the medical procedure), as a time series of values, or after the completion of the medical procedure or phase thereof. The data processing system 130 can display the performance metrics (e.g., performance metrics 162 displayed on graphical user interface 236 in FIG. 2B). The metric generator 142 can determine if the actual performance metrics associated with performance of the medical procedure meet or exceed the performance metrics from the simulation, and display a corresponding indication. For example, if the actual performance metrics are worse than the simulated performance metrics, the data processing system 130 can provide a warning, guidance, alarm, or other indication to cause the surgeon to more closely follow the simulated path or otherwise take corrective measures to improve the performance of the medical procedure. To perform the comparison, the data processing system 130 can compare values of performance metrics having the same or similar time stamps relative to a start time of the medical procedure. In some cases, to perform the comparison, the data processing system 130 can compare values of performance metrics based on the detected location of the actual medical tool in comparison to the simulated location of the medical tool. The data processing system 130, by generating values of performance metrics in real-time and comparing the values to the corresponding values of performance metrics generated during the simulation using the customized 3D model of the organ, can improve the performance of the actual medical procedure.

Thus, the data processing system 130 can receive, via one or more sensors, a data stream of the procedure performed via the robotic medical system on the subject in a medical environment. The data processing system 130 can determine a first value of a performance metric associated with the procedure performed via the robotic medical system on the subject in the medical environment. The data processing system 130 can determine a second value of the performance metric related to the candidate path identified during execution of the simulation. The data processing system 130 can provide a notification via the graphical user interface based on a comparison of the first value and the second value. The data processing system 130 can generate a value of a second performance metric based at least in part on the comparison of the first value and the second value. For example, the second performance metric can be a binary value that indicates whether the value of the first performance metric during the actual procedure is greater than or equal to the value of the first performance metric in the simulation, or less than the value of the first performance metric in simulation. The second performance metric can be a numeric value that indicates the amount of deviation between the value of the first performance metric during the medical comparison as compared to the value of the first performance metric in simulation. The numeric value of the second performance metric can be an absolute value, positive or negative value, or percentage, for example. The data processing system 130 can display, via the graphical user interface, the value of the second performance metric.

The tool navigator 144 can determine, based on the data stream, a location of the medical tool 120 during the medical procedure. The data stream can include visual information of the medical tool captured by a camera, and the data processing system 130 can display the video feed which can include the medical tool and the anatomical structure. In some cases, the data processing system 130 can determine the location of the tool using additional data stream elements, such as data from a kinematics stream which can indicate the position of the medical tool and orientation of the medical tool 120. The data processing system 130 can display the location of the medical tool via a graphical user interface. The data processing system 130 can display the location of the medical tool 120 together with the path generated for the medical tool during the simulation. For example, the data processing system 130 can overlay the actual location on the simulated path. The data processing system 130 can overlay the simulated path on the anatomical structure captured via the video stream, and display the actual location of the medical tool on the anatomical structure captured via the video stream. By displaying both an indication of the location of the actual medical tool and the simulated path, the data processing system 130 can display whether the surgeon is on track with the simulated path or deviated from the simulated path.

If the surgeon is deviating during the medical procedure from the simulated path, the data processing system 130 can determine a distance between the location of the tool 120 during the medical procedure and the candidate path for the tool. The data processing system 130 can provide a second notification responsive to the distance being greater than a threshold. For example, the threshold can be a distance threshold such as 5 millimeters, 10 millimeters, 15 millimeters, 30 millimeters, 50 millimeters, 1 centimeter, or other distance threshold. The data processing system 130 can select the threshold from the threshold data structure 164 based at least in part on the type of the procedure or the type of the organ. For example, the threshold can be relative to the size of the anatomical structure on which the medical procedure is to be performed (e.g., a smaller threshold for a smaller organ). If the deviation is greater than the threshold, the data processing system 130 can provide an audio or visual alert. The alert can indicate the amount of deviation.

FIG. 2A depicts an example method of tool navigation using a customized medical simulation. The method 200 can be performed by one or more system or component depicted in FIG. 1 or FIG. 6, including, for example, a data processing system. At ACT 202, a scanning device can scan a subject. The scanning device can be a CT machine or MRI machine, or other type of imaging machine. The scanning device can include an X-ray machine. The scan of the subject can include a multi-modal scan, such as a scan using multiple types of medical imaging techniques. The scan of the subject can be performed in the medical environment, or at a remote site separate from the medical environment. The scan can be localized or tailored for the anatomical structure on which the medical subject procedure is to be performed. For example, the scan can be performed for the organ on which the medical procedure is to be performed.

At ACT 204, the data processing system can receive medical images from the scan. The data processing system can receive the medical images via a network or a physical storage medium. The scan of the medical images can be associated with a subject identifier, time stamp, or metadata. The scan of the medical images can indicate metadata such as the anatomical structure identifier or label, type of scan, resolution of the scan, or number of images.

At ACT 206, the data processing system can create a 3D surgical model (e.g., 3D model 156). The data processing system can create the 3D surgical model based on the medical images from the scan received at ACT 204. The 3D model can include an organ and a tumor or cancer on the organ, for example. The data processing system can access a simulation library to create the 3D model. For example, the 3D model can leverage predetermined or previously established components available in the simulation library, such as texture information, color information, mesh surface information, dimensions, volume, underlying anatomical structures, or coupling points.

At ACT 210, the data processing system can register the model. The data processing system can register the model with a simulated anatomy obtained from the simulation library 208 or other information obtained from the historical case data 212. For example, the data processing system can leverage historical case data 212 to determine how to register the 3D model. The historical case data can include information about coupling points, orientation, and salient anatomical structures for the type of medical procedure. For example, for a nephrectomy, the historical case data can indicate which coupling points to use for the 3D model of the organ, and which parts of the simulated anatomy to maintain when performing the registration.

At ACT 214, the data processing system can establish a customized simulation. The data processing system can establish a digital environment that includes the 3D model registered or overlayed on the simulated anatomy from the simulation library 208. The data processing system can establish the customized simulation with objects such as a simulated medical tool. The data processing system can obtain the simulated medical tool from the simulation library 208. The data processing system can establish the simulation with interactive features, such that the simulation is interactive. For example, the data processing system can establish a physics engine for the simulation in order to provide realistic feedback.

At ACT 216, the data processing system can execute the customized, interactive simulation. Executing the simulation can include a user or surgeon interacting with the simulation in order to perform a simulated medical procedure. The surgeon can interact with the simulation using input devices, such as manipulators, keyboard, mouse, joystick, scroll ball, or other input devices that can control or move a simulated medical tool in the simulation.

At ACT 218, the data processing system can generate performance metrics for the simulation. Performance metrics, or objective performance indicators, can be generated based on the interactions carried out during the simulation. The performance metrics can be generated in real-time during the simulation, or upon completion of the simulation, depending on the type of performance metric.

At decision block 220, the data processing system can determine whether the performance of the simulated medical procedure was satisfactory. For example, the data processing system can compare the values of the performance metrics generated at ACT 218 with thresholds established for the medical procedure. If the values satisfy the thresholds, then the data processing system can proceed to ACT 222. If however, the values do not satisfy the threshold, the data processing system can return to ACT 216 to initiated another iteration of the simulation to allow the surgeon to improve the performance of the medical procedure. For example, the surgeon may improve the skill with which the procedure is performed, or identify a different candidate path for the medical tool that results in improved performance metrics (e.g., increases the amount of organ that is remaining, or eliminate positive margin).

At ACT 222, the data processing system can identify parameters of the simulation that results in the satisfactory performance. The parameters can include, for example, the path the medical tool followed during the simulated medical procedures. Parameters can include the angle of the medical tool, kinematics information about the medical tool, type of the medical tool, relative angles between multiple medical tools, or other information that can facilitate guiding the surgeon, during the actual medical procedure, to repeat the satisfactory performance.

At ACT 224, the data processing system can provide the identified parameter to perform the surgical procedure. For example, the data processing system can display the parameter on a graphical user interface before or during the medical procedure. The data processing system can display a path or other parameter to allow the surgeon to follow the same path during the medical procedure.

FIG. 2B depicts example graphical user interface (“GUI”) visualization for tool navigation using a customized medical simulation. The visualization elements can be used by or generated by one or more system or component depicted in FIG. 1 or FIG. 6, including, for example, a data processing system. For example, FIG. 2B depicts scan data 154, which can correspond to scans from a medical imaging technique. FIG. 2B depicts a 3D model 156 generated by the data processing system. The 3D model 156 (or 3D surgical model) can include a cancer 232. The cancer 232 can be part of the scan data 154. The data processing system can convert the scan data 154 to the 3D medical model 156, and include the cancer 232 in the event the medical procedure relates to removing the cancer 232. The simulation library 166 can include various simulated anatomies 152 or simulated medical tools 234. The data processing system can include the 3D model 156, cancer 232, simulated anatomy 152, and simulated medical tool 234 from the simulation library 166 in the digital environment to create the simulation. The data processing system can register or overlay the 3D model 156 with or on the simulated anatomy 152.

The surgeon can use the user control system 510 to interact with the simulation or perform the medical procedure. For example, during the medical procedure or the simulation, the user control system 510 can provide information via a graphical user interface 236 displayed on a display device 630. The graphical user interface 236 can display the organ 242 (e.g., the actual organ from a video stream or the 3D model 156 if a simulation). The GUI 236 can display an overlay of the path 238. The GUI 236 can display a video stream of the actual medical procedure 240. The data processing system can display performance metrics 162 on the GUI 236.

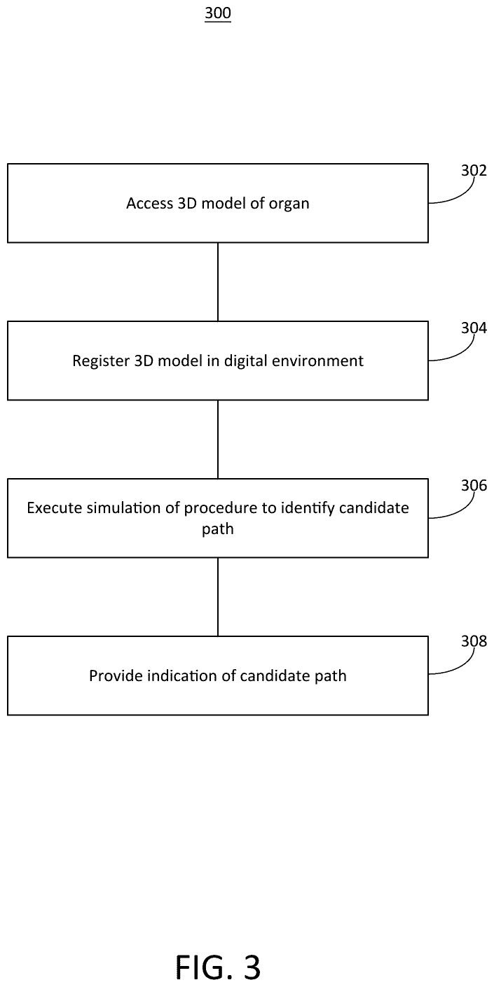

FIG. 3 depicts an example method of tool navigation using a customized medical simulation. The method 300 can be performed by one or more system or component depicted in FIG. 1 or FIG. 6, including, for example, a data processing system. At ACT 302, the data processing system can access the 3D model of the organ. The data processing system can obtain the 3D model of the organ from a data repository. The data processing system can perform a lookup based on a patient identifier or other unique identifier to obtain the 3D model of the organ. At ACT 304, the data processing system can register the 3D model in a digital environment. The data processing system can obtain additional simulated anatomical structures or objects from a simulation library in order to register the 3D model. For example, the data processing system can obtain simulated anatomies that are proximate to the organ and simulated medical tools that may be used to perform the medical procedure. The data processing system can receive an indication from the user of which simulated medical tools to include, or the data processing system can automatically determine or select the medical tools to include based on the type of the medical procedure or the type of the organ. For example, the data processing system can leverage case history information to determine which medical tools were used historically to perform the type of medical procedure, and automatically create the digital environment to include the selected medical tools.

At ACT 306, the data processing system can execute the simulation of the procedure to identify the candidate path. The user or surgeon can interact with the simulation to control a simulated medical tool to perform the medical procedure, and the data processing system can record the path or other parameters associated with the simulated medical procedure. At ACT 308, the data processing system can provide an indication of the candidate path. The data processing system can provide the indication of the path or other parameters during the simulation or upon completion of the simulation. For example, the data processing system can provide a real-time display of the parameter (e.g., path generated by the medical tool) via a graphical user interface.

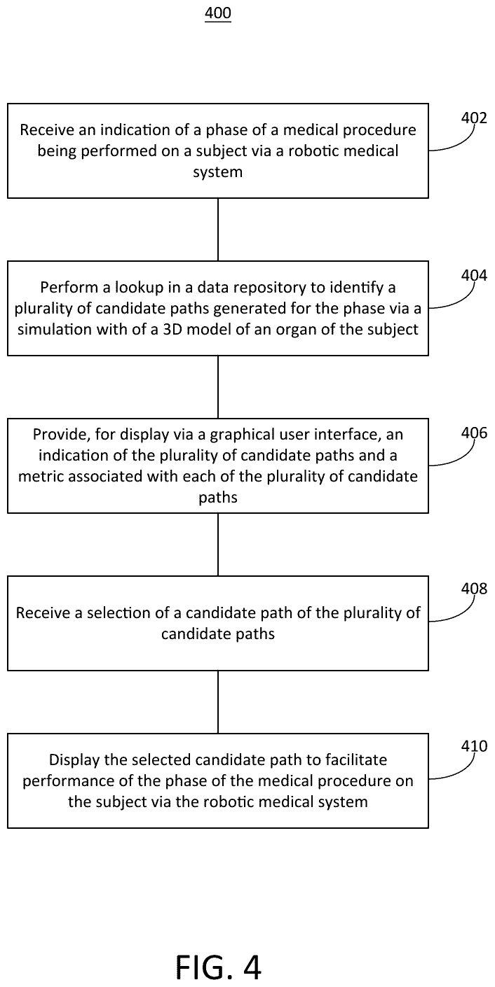

FIG. 4 depicts an example method of tool navigation using a customized medical simulation. The method 400 can be performed by one or more system or component depicted in FIG. 1 or FIG. 6, including, for example, a data processing system. The data processing system can have different simulated paths for different phases of the medical procedure. The data processing system can select a path based on the phase of the medical procedure. For example, at ACT 402, the data processing system can receive an indication of a phase of the medical procedure being performed on a subject via a robotic medical system. The data processing system can receive the indication via user input, such as through a user interface or input device. In some cases, the data processing system can automatically determine the phase of the medical procedure based on a data stream, such as type of medical tool installed on the robotic medical system 124, the tasks or gestures being performed, the anatomical structures that are present in a video stream, or a workflow (e.g., order of phases) established for the type of medical procedure.

Upon detecting the phase of the medical procedure, the data processing system can select a path for the phase at ACT 404. The data processing system can perform a lookup in a data repository to identify a plurality of candidate paths generated for the phase via a simulation with of a 3D model of an organ of the subject. The candidate paths stored in the data repository can each be associated with a phase of the medical procedure, along with corresponding performance metrics. The data processing system can automatically select a highest scoring candidate path for a particular phase of the medical procedure.

At ACT 406, the data processing system can provide, for display via a graphical user interface, an indication of the plurality of candidate paths and a metric associated with each of the plurality of candidate paths. The data processing system can provide can display the current phase of the medical procedure, and provide the available candidate paths generated via the simulation for the same phase of the medical procedure. The data processing system can rank the candidate paths, or provide indications of the overall scores or particular performance metrics for each candidate path.

At ACT 408, the data processing system can receive a selection of the candidate path for the phase. For example, a user can provide input via a user interface to select a path from the multiple available paths for the particular phase of the medical procedure. At ACT 410, the data processing system can display the selected candidate path to facilitate performance of the phase of the medical procedure on the subject via the robotic medical system.

FIG. 5 depicts an example medical environment 104, in accordance with embodiments. The medical environment 104 can refer to or include a surgical environment or surgical system. The medical environment 104 can include a robotic medical system 124, a user control system 510, and an auxiliary system 515 communicatively coupled one to another. A visualization tool 520 (e.g., the visualization tool 170) can be connected to the auxiliary system 515, which in turn can be connected to the robotic medical system 124. Thus, when the visualization tool 520 is connected to the auxiliary system 515 and this auxiliary system is connected to the robotic medical system 124, the visualization tool can be considered connected to the robotic medical system. In some embodiments, the visualization tool 520 can additionally or alternatively be directly connected to the robotic medical system 124.

The medical environment 104 can be used to perform a computer-assisted medical procedure with a patient 525. In some embodiments, surgical team can include a surgeon 530A and additional medical personnel 530B-530D such as a medical assistant, nurse, and anesthesiologist, and other suitable team members who can assist with the surgical procedure or medical session. The medical session can include the surgical procedure being performed on the patient 525, as well as any pre-operative (e.g., which can include setup of the medical environment 104, including preparation of the patient 525 for the procedure), and post-operative (e.g., which can include clean up or post care of the patient), or other processes during the medical session. Although described in the context of a surgical procedure, the medical environment 104 can be implemented in a non-surgical procedure, or other types of medical procedures or diagnostics that can benefit from the accuracy and convenience of the surgical system.

The robotic medical system 124 can include a plurality of manipulator arms 535A-535D to which a plurality of medical tools (e.g., the medical tool 120) can be coupled or installed. Each medical tool can be any suitable surgical tool (e.g., a tool having tissue-interaction functions), imaging device (e.g., an endoscope, an ultrasound tool, etc.), sensing instrument (e.g., a force-sensing surgical instrument), diagnostic instrument, or other suitable instrument that can be used for a computer-assisted surgical procedure on the patient 525 (e.g., by being at least partially inserted into the patient and manipulated to perform a computer-assisted surgical procedure on the patient). Although the robotic medical system 124 is shown as including four manipulator arms (e.g., the manipulator arms 535A-535D), in other embodiments, the robotic medical system can include greater than or fewer than four manipulator arms. Further, not all manipulator arms can have a medical tool installed thereto at all times of the medical session. Moreover, in some embodiments, a medical tool installed on a manipulator arm can be replaced with another medical tool as suitable.

One or more of the manipulator arms 535A-535D or the medical tools attached to manipulator arms can include one or more displacement transducers, orientational sensors, positional sensors, or other types of sensors and devices to measure parameters or generate kinematics information. One or more components of the medical environment 104 can be configured to use the measured parameters or the kinematics information to track (e.g., determine poses of) or control the medical tools, as well as anything connected to the medical tools or the manipulator arms 535A-535D.

The user control system 510 can be used by the surgeon 530A to control (e.g., move) one or more of the manipulator arms 535A-535D or the medical tools connected to the manipulator arms. To facilitate control of the manipulator arms 535A-535D and track progression of the medical session, the user control system 510 can include a display (e.g., the display 172) that can provide the surgeon 530A with imagery (e.g., high-definition 3D imagery) of a surgical site associated with the patient 525 as captured by a medical tool (e.g., the medical tool 120, which can be an endoscope) installed to one of the manipulator arms 535A-535D. The user control system 510 can include a stereo viewer having two or more displays where stereoscopic images of a surgical site associated with the patient 525 and generated by a stereoscopic imaging system can be viewed by the surgeon 530A. In some embodiments, the user control system 510 can also receive images from the auxiliary system 515 and the visualization tool 520.

The surgeon 530A can use the imagery displayed by the user control system 510 to perform one or more procedures with one or more medical tools attached to the manipulator arms 535A-535D. To facilitate control of the manipulator arms 535A-535D or the medical tools installed thereto, the user control system 510 can include a set of controls. These controls can be manipulated by the surgeon 530A to control movement of the manipulator arms 535A-535D or the medical tools installed thereto. The controls can be configured to detect a wide variety of hand, wrist, and finger movements by the surgeon 530A to allow the surgeon to intuitively perform a procedure on the patient 525 using one or more medical tools installed to the manipulator arms 535A-535D.

The auxiliary system 515 can include one or more computing devices configured to perform processing operations within the medical environment 104. For example, the one or more computing devices can control or coordinate operations performed by various other components (e.g., the robotic medical system 124, the user control system 510) of the medical environment 104. A computing device included in the user control system 510 can transmit instructions to the robotic medical system 124 by way of the one or more computing devices of the auxiliary system 515. The auxiliary system 515 can receive and process image data representative of imagery captured by one or more imaging devices (e.g., medical tools) attached to the robotic medical system 124, as well as other data stream sources received from the visualization tool. For example, one or more image capture devices (e.g., the data capture devices 110) can be located within the medical environment 104. These image capture devices can capture images from various viewpoints within the medical environment 104. These images (e.g., video streams) can be transmitted to the visualization tool 520, which can then passthrough those images to the auxiliary system 515 as a single combined data stream. The auxiliary system 515 can then transmit the single video stream (including any data stream received from the medical tool(s) of the robotic medical system 124) to present on a display (e.g., the display 172) of the user control system 510.

In some embodiments, the auxiliary system 515 can be configured to present visual content (e.g., the single combined data stream) to other team members (e.g., the medical personnel 530B-530D) who may not have access to the user control system 510. Thus, the auxiliary system 515 can include a display 640 configured to display one or more user interfaces, such as images of the surgical site, information associated with the patient 525 or the surgical procedure, or any other visual content (e.g., the single combined data stream). In some embodiments, display 640 can be a touchscreen display or include other features to allow the medical personnel 530A-530D to interact with the auxiliary system 515.

The robotic medical system 124, the user control system 510, and the auxiliary system 515 can be communicatively coupled one to another in any suitable manner. For example, in some embodiments, the robotic medical system 124, the user control system 510, and the auxiliary system 515 can be communicatively coupled by way of control lines 645, which can represent any wired or wireless communication link as can serve a particular implementation. Thus, the robotic medical system 124, the user control system 510, and the auxiliary system 515 can each include one or more wired or wireless communication interfaces, such as one or more local area network interfaces, Wi-Fi network interfaces, cellular interfaces, etc.

It is to be understood that the medical environment 104 can include other or additional components or elements that can be needed or considered desirable to have for the medical session for which the surgical system is being used.

FIG. 6 is a block diagram depicting an architecture for a computer system 600 that can be employed to implement elements of the systems and methods described and illustrated herein, including aspects of the systems depicted in FIG. 1 or FIG. 5, and the methods or processes depicted in FIGS. 3 and 4. For example, the data processing system 130, robotic medical system 124, or client device 174 can include one or more component or functionality of computing system 600. The computer system 600 can be any computing device used herein and can include or be used to implement a data processing system or its components. The computer system 600 includes at least one bus 605 or other communication component or interface for communicating information between various elements of the computer system. The computer system further includes at least one processor 610 or processing circuit coupled to the bus 605 for processing information. The computer system 600 also includes at least one main memory 615, such as a random-access memory (RAM) or other dynamic storage device, coupled to the bus 605 for storing information, and instructions to be executed by the processor 610. The main memory 615 can be used for storing information during execution of instructions by the processor 610. The computer system 600 can further include at least one read only memory (ROM) 620 or other static storage device coupled to the bus 605 for storing static information and instructions for the processor 610. A storage device 625, such as a solid-state device, magnetic disk or optical disk, can be coupled to the bus 605 to persistently store information and instructions.

The computer system 600 can be coupled via the bus 605 to a display 630, such as a liquid crystal display, or active-matrix display, for displaying information. An input device 635, such as a keyboard or voice interface can be coupled to the bus 605 for communicating information and commands to the processor 610. The input device 635 can include a touch screen display (e.g., the display 630). The input device 635 can include sensors to detect gestures. The input device 635 can also include a cursor control, such as a mouse, a trackball, or cursor direction keys, for communicating direction information and command selections to the processor 610 and for controlling cursor movement on the display 630.

The processes, systems and methods described herein can be implemented by the computer system 600 in response to the processor 610 executing an arrangement of instructions contained in the main memory 615. Such instructions can be read into the main memory 615 from another computer-readable medium, such as the storage device 625. Execution of the arrangement of instructions contained in the main memory 615 causes the computer system 600 to perform the illustrative processes described herein. One or more processors in a multi-processing arrangement can also be employed to execute the instructions contained in the main memory 615. Hard-wired circuitry can be used in place of or in combination with software instructions together with the systems and methods described herein. Systems and methods described herein are not limited to any specific combination of hardware circuitry and software.

The processor 610 can execute one or more instructions associated with the system 100. The processor 610 can include an electronic processor, an integrated circuit, or the like including one or more of digital logic, analog logic, digital sensors, analog sensors, communication buses, volatile memory, nonvolatile memory, and the like. The processor 610 can include, but is not limited to, at least one microcontroller unit (MCU), microprocessor unit (MPU), central processing unit (CPU), graphics processing unit (GPU), physics processing unit (PPU), embedded controller (EC), or the like. The processor 610 can include, or be associated with, a memory 615 operable to store or storing one or more non-transitory computer-readable instructions for operating components of the system 100 and operating components operably coupled to the processor 610. The one or more instructions can include at least one of firmware, software, hardware, operating systems, or embedded operating systems, for example. The processor 610 or the system 100 generally can include at least one communication bus controller to effect communication between the system processor and the other elements of the system 100.

The memory 615 can include one or more hardware memory devices to store binary data, digital data, or the like. The memory 615 can include one or more electrical components, electronic components, programmable electronic components, reprogrammable electronic components, integrated circuits, semiconductor devices, flip flops, arithmetic units, or the like. The memory 615 can include at least one of a non-volatile memory device, a solid-state memory device, a flash memory device, a NAND memory device, a volatile memory device, etc. The memory 615 can include one or more addressable memory regions disposed on one or more physical memory arrays.

Although an example computing system has been described in FIG. 6, the subject matter including the operations described in this specification can be implemented in other types of digital electronic circuitry, or in computer software, firmware, or hardware, including the structures disclosed in this specification and their structural equivalents, or in combinations of one or more of them.

The herein described subject matter sometimes illustrates different components contained within, or connected with, different other components. It is to be understood that such depicted architectures are illustrative, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable or physically interacting components or wirelessly interactable or wirelessly interacting components or logically interacting or logically interactable components.

With respect to the use of plural or singular terms herein, those having skill in the art can translate from the plural to the singular or from the singular to the plural as is appropriate to the context or application. The various singular/plural permutations can be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

Although the figures and description can illustrate a specific order of method steps, the order of such steps can differ from what is depicted and described, unless specified differently above. Also, two or more steps can be performed concurrently or with partial concurrence, unless specified differently above. Such variation can depend, for example, on the software and hardware systems chosen and on designer choice. All such variations are within the scope of the disclosure. Likewise, software implementations of the described methods can be accomplished with standard programming techniques with rule-based logic and other logic to accomplish the various connection steps, processing steps, comparison steps, and decision steps.

It will be further understood by those within the art that if a specific number of an introduced claim recitation is intended, such an intent will be explicitly recited in the claim, and in the absence of such recitation, no such intent is present. For example, as an aid to understanding, the following appended claims can contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, or A, B, and C together, etc.). In those instances where a convention analogous to “at least one of A, B, or C, etc.” is used, in general, such a construction is intended in the sense one having skill in the art would understand the convention (e.g., “a system having at least one of A, B, or C” would include but not be limited to systems that have A alone, B alone, C alone, A and B together, A and C together, B and C together, or A, B, and C together, etc.). It will be further understood by those within the art that virtually any disjunctive word or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Further, unless otherwise noted, the use of the words “approximate,” “about,” “around,” “substantially,” etc., mean plus or minus ten percent.

The foregoing description of illustrative implementations has been presented for purposes of illustration and of description. It is not intended to be exhaustive or limiting with respect to the precise form disclosed, and modifications and variations are possible in light of the above teachings or can be acquired from practice of the disclosed implementations. It is intended that the scope of the invention be defined by the claims appended hereto and their equivalents.

What is claimed is:

1. A system, comprising:
one or more processors, coupled with memory, to:
access a 3-dimensional model of an anatomical structure generated via a scan of a subject on which a procedure is to be performed via a robotic medical system;
register the 3-dimensional model of the anatomical structure with predetermined coupling points in a digital environment established for the procedure on the subject;
execute, via the digital environment, a simulation of the procedure to identify a candidate path for a tool through the 3-dimensional model of the anatomical structure registered with the predetermined coupling points in the digital environment; and
provide, for display via a graphical user interface, an indication of the candidate path for the tool to perform the procedure via the robotic medical system.

2. The system of claim 1, wherein the one or more processors are further configured to:
access a 3-dimensional model of the subject generated with a first resolution, wherein the 3-dimensional model of the anatomical structure is generated with a second resolution that is greater than the first resolution; and
register, via the predetermined coupling points, the 3-dimensional model of the anatomical structure with the 3-dimensional model of the subject in the digital environment.

3. The system of claim 1, wherein the one or more processors are further configured to:
receive an indication of one or more portions of the subject on which the procedure is to be performed;
identify a resolution of one or more 3-dimensional models for the one or more portions of the subject based on the indication that the procedure is to be performed on the one or more portions; and
select the 3-dimensional models having the resolution for registration in the digital environment.

4. The system of claim 1, wherein the anatomical structure comprises an organ, the predetermined coupling points comprise a first vein and a second vein, and the one or more processors are further configured to:
align a first point on the 3-dimensional model of the organ with the first vein in the digital environment; and
align a second point on the 3-dimensional model of the organ with the second vein in the digital environment.

5. The system of claim 1, wherein the one or more processors are further configured to:
adjust a size of a simulated anatomy in the digital environment to register the 3-dimensional model of the anatomical structure in the digital environment.

6. The system of claim 1, wherein the one or more processors are further configured to:
adjust an orientation of the 3-dimensional model of the anatomical structure to register the 3-dimensional model of the anatomical structure in the digital environment.

7. The system of claim 1, wherein the one or more processors are further configured to:
identify, upon execution of the simulation of the procedure, a plurality of candidate paths for the tool through the 3-dimensional model, wherein the plurality of candidate paths comprises the candidate path;
determine, based on the simulation, a first value for a performance metric for the candidate path of the plurality of candidate paths;
determine, based on the simulation, a second value for the performance metric for a second candidate path of the plurality of candidate paths;
select the candidate path based on a comparison of the first value and the second value; and
provide, responsive to the selection, the indication for the candidate path for display via the graphical user interface.

8. The system of claim 1, wherein the one or more processors are further configured to:
identify, upon execution of the simulation of the procedure, a value of a performance metric related to the candidate path for the tool to perform;
identify a threshold for the performance metric based on at least one of a type of procedure or a type of the anatomical structure; and
provide, responsive to the value of the performance metric satisfying the threshold, the candidate path for display via the graphical user interface.

9. The system of claim 8, wherein the one or more processors are further configured to:
display, via the graphical user interface, one or more values of the performance metric in association with one or more candidate paths.

10. The system of claim 1, wherein the one or more processors are further configured to:
overlay, via the graphical user interface, the indication of the candidate path on the 3-dimensional model of the anatomical structure.

11. The system of claim 1, wherein the one or more processors are further configured to:
receive a video stream captured by a camera of the procedure performed via the robotic medical system on the subject in a medical environment;
display the video stream via the graphical user interface; and
overlay the indication of the candidate path for the tool through the anatomical structure on the video stream of the procedure displayed via the graphical user interface.

12. The system of claim 11, wherein the one or more processors are further configured to:
receive, subsequent to the display of the candidate path, a command to reject the candidate path displayed via the graphical user interface;
remove, responsive to the command, the display of the candidate path; and
display, responsive to the command, a second candidate path identified during execution of the simulation.

13. The system of claim 1, wherein the one or more processors are further configured to:
receive, via one or more sensors, a data stream of the procedure performed via the robotic medical system on the subject in a medical environment;
determine a first value of a performance metric associated with the procedure performed via the robotic medical system on the subject in the medical environment;
determine a second value of the performance metric related to the candidate path identified during execution of the simulation; and
provide a notification via the graphical user interface based on a comparison of the first value and the second value.

14. The system of claim 13, wherein the one or more processors are further configured to:
generate a value of a second performance metric based at least in part on the comparison of the first value and the second value; and
display, via the graphical user interface, the value of the second performance metric.

15. The system of claim 13, wherein the one or more processors are further configured to:
display a location of the tool during the procedure performed via the robotic medical system on the subject in the medical environment; and
display the location of the tool on the graphical user interface comprising the indication of the candidate path for the tool to perform the procedure via the robotic medical system.

16. The system of claim 15, wherein the one or more processors are further configured to:
determine a distance between the location of the tool and the candidate path for the tool; and
provide a second notification responsive to the distance being greater than a threshold.

17. The system of claim 16, wherein the threshold is selected based at least in part on a type of the procedure or the type of the anatomical structure.

18. A method, comprising:
registering, by one or more processors coupled with memory, a 3-dimensional model of an organ with predetermined coupling points in a digital environment established for a procedure to be performed via robotic medical system on a subject;
simulating, by the one or more processors via the digital environment, the procedure to identify a candidate path for a tool through the 3-dimensional model of the organ registered with the predetermined coupling points in the digital environment; and
providing, by the one or more processors for display via a graphical user interface, an indication of the candidate path for the tool to perform the procedure via the robotic medical system.

19. The method of claim 18, comprising:
accessing, by the one or more processors, a 3-dimensional model of the subject generated with a first resolution, wherein the 3-dimensional model of the organ is generated with a second resolution that is greater than the first resolution; and
registering, by the one or more processors via the predetermined coupling points, the 3-dimensional model of the organ with the 3-dimensional model of the subject in the digital environment.

20. A non-transitory computer-readable medium storing processor-executable instructions that, when executed by one or more processors, cause the one or more processors to:
download a 3-dimensional model of an organ generated via a scan of a subject on which a procedure is to be performed via a robotic medical system;
overlay the 3-dimensional model of the organ with predetermined coupling points in a digital environment established for the procedure on the subject;
execute, via the digital environment, a simulation of the procedure to identify a candidate path for a tool through the 3-dimensional model of the organ registered with the predetermined coupling points in the digital environment; and
display, via a graphical user interface, an indication of the candidate path for the tool to perform the procedure via the robotic medical system.
